(19) United States
(12) Patent Application Publication
(10) Pub. No.: US 2025/0281379 A1
(43) Pub. Date: Sep. 11, 2025

(54) Cosmetic and Personal Care compositions

(71) Applicant: BOLT THREADS, INC., Emeryville, CA (US)

(72) Inventors: David Breslauer, Emeryville, CA (US); Lindsay Wray Wray, Benicia, CA (US); Chelsi Oestreich, Emeryville, CA (US); Cintia Nardi, Emeryville, CA (US)

(21) Appl. No.: 19/060,145

(22) Filed: Feb. 21, 2025

Related U.S. Application Data
(60) Provisional application No. 63/556,170, filed on Feb. 21, 2024.

(51) Int. Cl. A61K 8/64; A61K 8/04; A61Q 1/02

(52) U.S. Cl. CPC A61K 8/64; A61K 8/042; A61Q 1/02; A61K 2800/43

(57) ABSTRACT

Cosmetic and/or personal care compositions can include a cosmetic or personal care base composition with a recombinant silk protein mixed therein, wherein the presence of the recombinant silk protein improves performance properties of the cosmetic and/or personal care composition.

### CROSS-REFERENCE TO RELATED APPLICATION

The benefit of priority to U.S. Provisional Patent Application No. 63/556,170 filed Feb. 21, 2024, is hereby claimed and the disclosure is incorporated herein by reference in its entirety.

### INCORPORATION BY REFERENCE OF THE SEQUENCE LISTING

The application contains, as a separate part of disclosure, a Sequence Listing in computer-readable form (Filename: 59877_SeqListing.xml; Size: 39,312 bytes; Created: May 30, 2025) which is incorporated by reference herein in its entirety.

### STATEMENT OF COLOR DRAWINGS

The patent or application file contains at least one drawing executed in color. Copies of this patent or patent application publication with color drawing(s) will be provided by the Office upon request and payment of the necessary fee.

### FIELD

The disclosure relates to compositions containing recombinant silk polypeptides for use in beauty and personal care.

### BACKGROUND

Proteins, such as silk proteins, have a variety of uses in cosmetic and personal care compositions. Natural proteins can be difficult to incorporate into formulations and/or provide only limited functionality, where multifunctional components are needed.

### SUMMARY

A need in the art exists for improved incorporation proteins in cosmetic and personal care compositions. Compositions of the disclosure can include a recombinant silk protein. Compositions can be cosmetic and/or personal care compositions. For example, compositions can be for use on skin, hair, teeth, or nails. Other compositions, such as household compositions, are also contemplated herein. The compositions of the disclosure can be in various forms, such as, but not limited, gels, liquids, lotions, suspensions, dispersions, creams, microemulsions, nano dispersions, microspheres, hydrogels, emulsions, and powders.

### BRIEF DESCRIPTION OF THE DRAWINGS

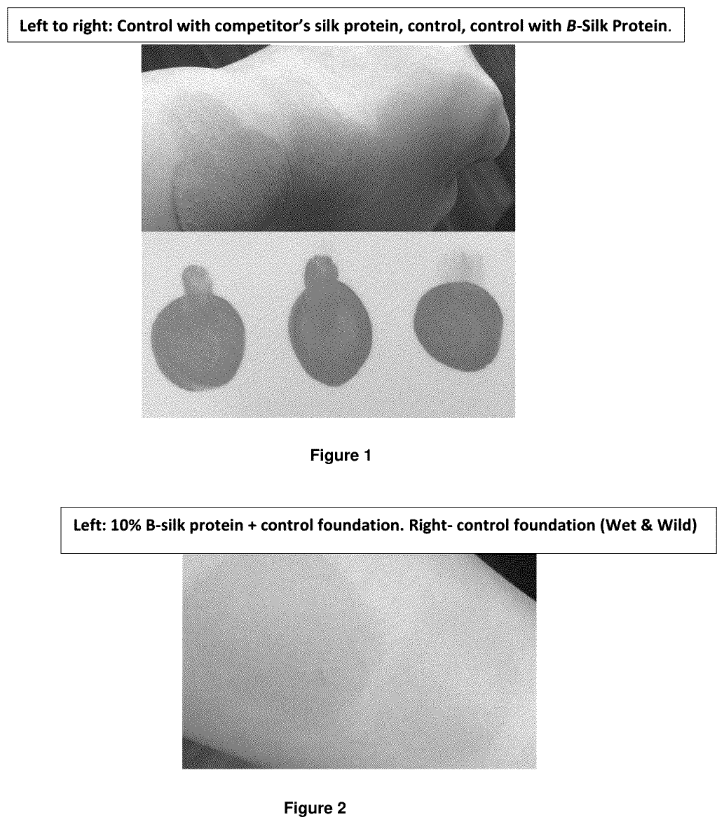

FIG. 1 is an image of comparative testing of a formulation in accordance with the disclosure with a conventional competitor's silk protein formulation and a control with no silk protein.

FIG. 2 is an image showing the comparison of a foundation with and without a recombinant silk protein in accordance with the disclosure.

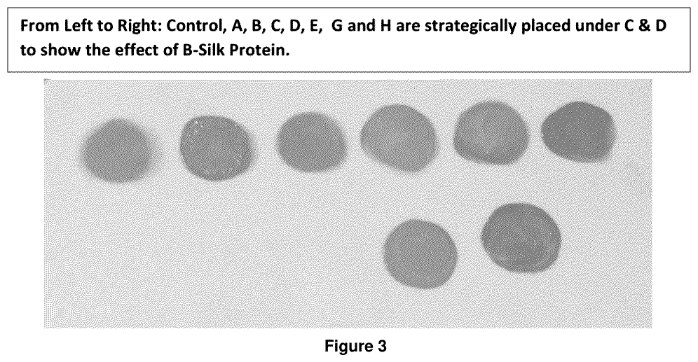

FIG. 3 is an image showing a comparison of control formulations without a recombinant silk protein to the formulations with a recombinant silk protein in accordance with the disclosure.

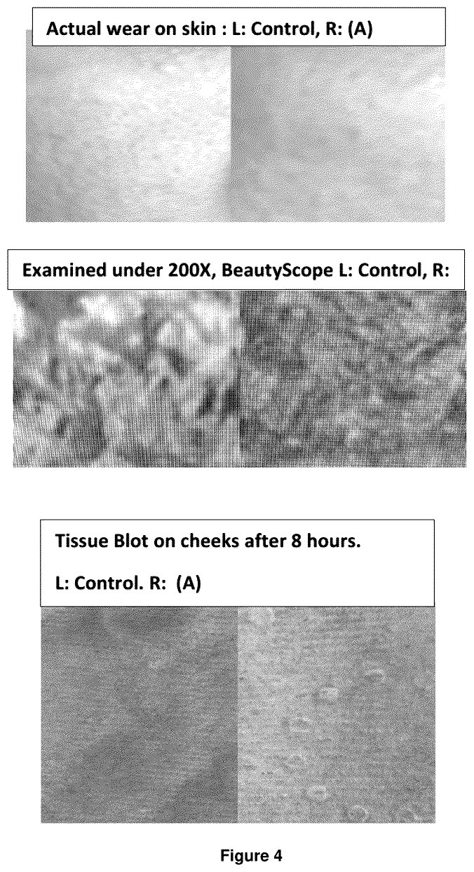

FIG. 4 are images showing a comparison of a control formulation without a recombinant silk protein to a formulation in accordance with the disclosure having a recombinant silk protein, illustrating improved wear achieved by the formulation in accordance with the disclosure.

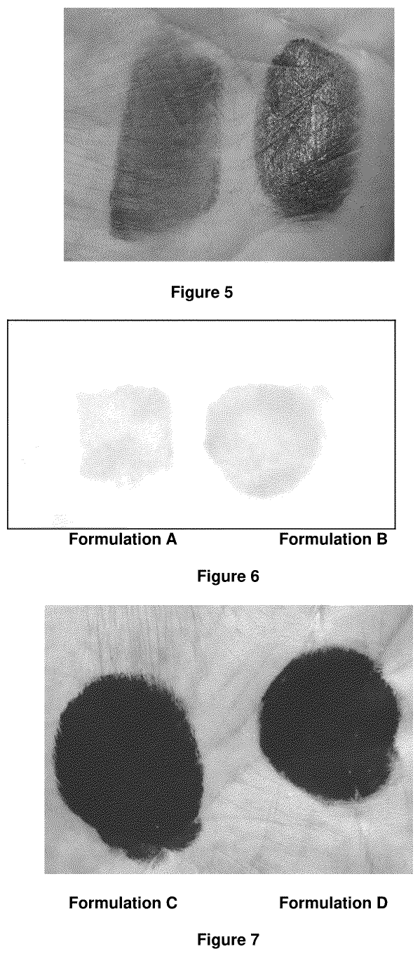

FIG. 5 is an image showing a comparison of a lipstick formulation control (left) and a formulation having a recombinant silk protein in accordance with the disclosure.

FIG. 6 is a photograph showing a comparison of a control to a formulation in accordance with the disclosure after a double cleaning.

FIG. 7 is a comparison of a control lipstick formulation to a formulation in accordance with the disclosure having a recombinant silk protein.

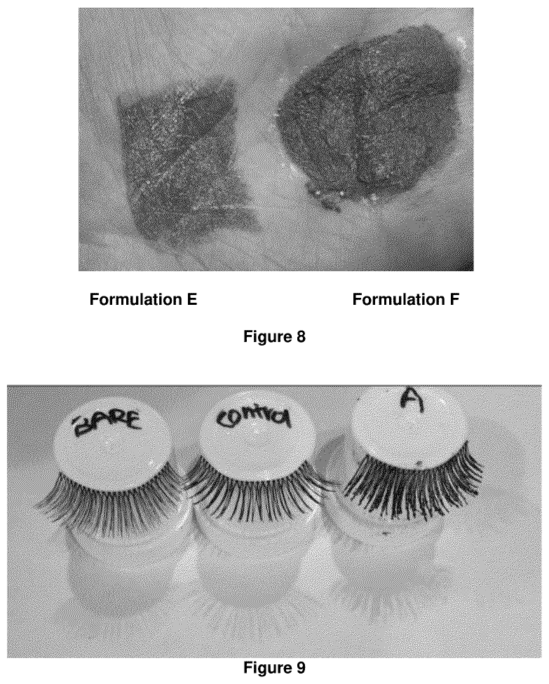

FIG. 8 is a comparison of a control lipstick formulation to a formulation in accordance with the disclosure having a recombinant silk protein.

FIG. 9 is a comparison of a control mascara to a formulation in accordance with the disclosure having a recombinant silk protein.

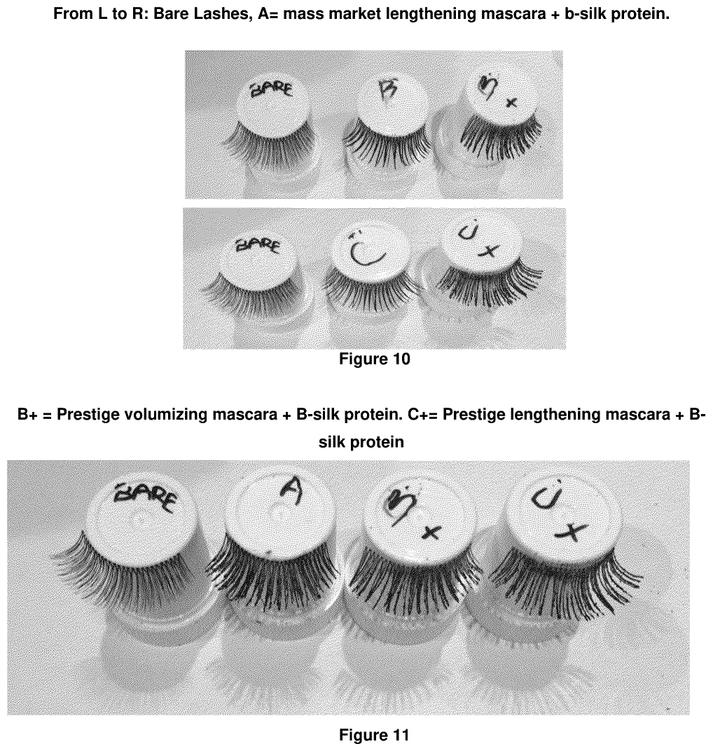

FIG. 10 shows comparisons of control mascaras to formulations in accordance with the disclosure having a recombinant silk protein.

FIG. 11 shows FIG. 9 shows comparisons of control mascaras to formulations in accordance with the disclosure having a recombinant silk protein.

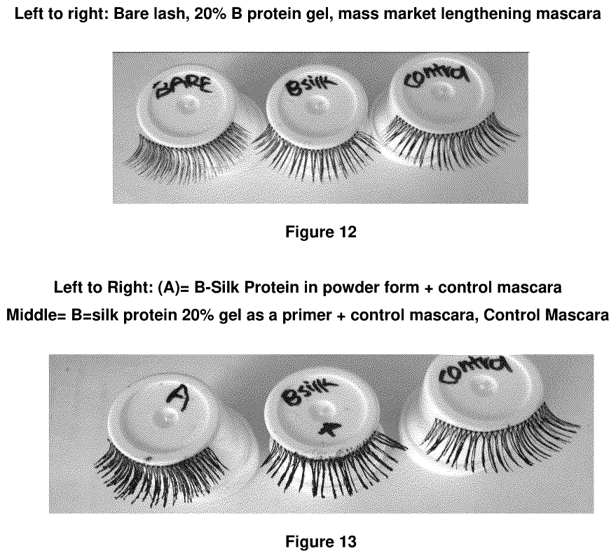

FIG. 12 is a comparison showing a 20% gel formulation of a recombinant silk protein in accordance with the disclosure to bare lashes and a control mascara.

FIG. 13 is a comparison of a 20% gel formulation used as a primer to a control formulation without a primer.

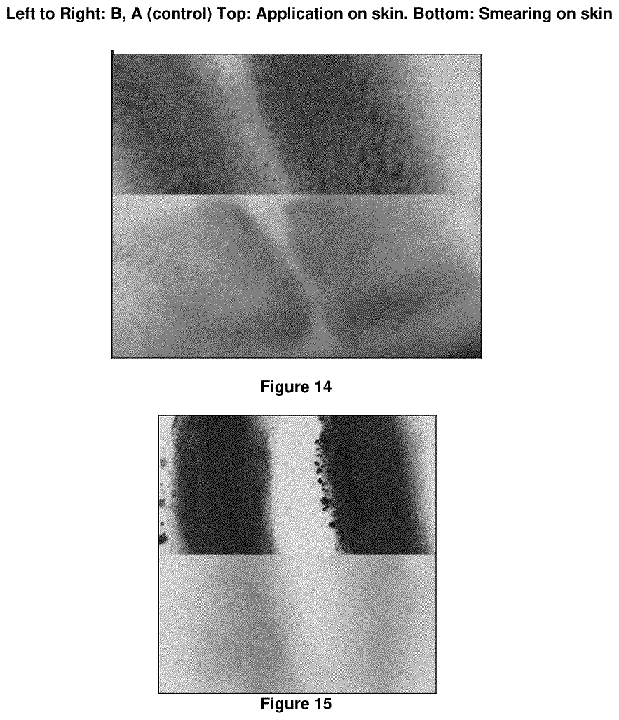

FIG. 14 is an image comparing eyeshadow formulations with a recombinant silk protein in accordance with the disclosure and without the protein (control).

FIG. 15 is an image comparing staying power of the formulation in accordance with the disclosure to the control.

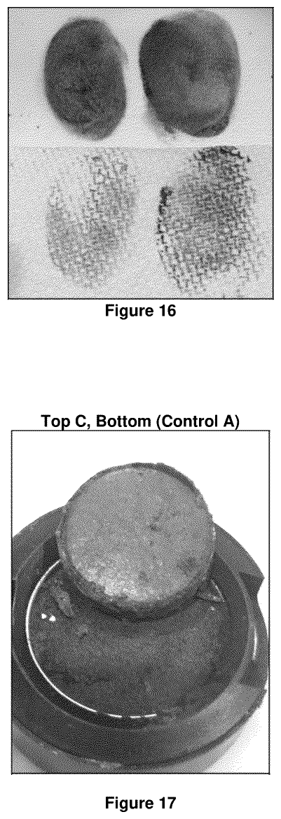

FIG. 16 is an image comparing a simulated eyelid creasing and performance of the eyeshadow formulations with a recombinant silk protein in accordance with the disclosure and without the protein (control).

FIG. 17 is an image of a repressed composition after addition of a recombinant silk protein in accordance with the disclosure and a comparison to the original formulation before repressing.

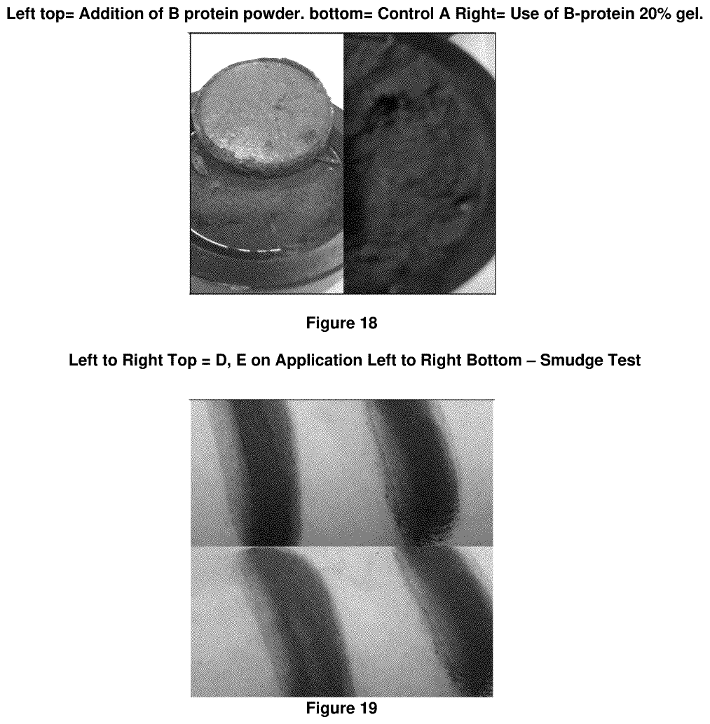

FIG. 18 is an image of a repressed composition after addition of a recombinant silk protein in a gel form in accordance with the disclosure and a comparison to the original formulation before repressing.

FIG. 19 is an image comparing the wear performance of a formulation with recombinant silk protein in the disclosure and the formulation without (control).

### DETAILED DESCRIPTION

Cosmetic, hair, or skin care compositions in accordance with the disclosure include a recombinant silk polypeptide. Compositions can further include one or more active ingredients for cosmetic, skin, or hair care.

Recombinant silk polypeptide is a high molecular weight polypeptide that has entropically self-assembled into a cross-linked and semicrystalline state. The recombinant polypeptide can be included in the composition as a powder. For example, hollow particles of recombinant silk polypeptide can be milled and included in the composition as a milled powder. The recombinant silk containing component can include the recombinant silk polypeptide present suspended in a solvent. The recombinant silk polypeptide can be present as a randomly structure gel. At the macro-level this could range from a low viscosity weak gel suspended within an aqueous solvent (sometimes referred to as “slurry”), to a dry hollow powder particle (<15% moisture content).

In some embodiments, provided herein is a composition comprising recombinant silk polypeptide and use thereof in beauty and personal care formulations.

In some embodiments, the recombinant silk polypeptide is a high molecular weight polypeptide greater than >100 amino acids in length and less than 90 kDa amino acids in length. For example, the recombinant silk protein can be commercially available B-Silk protein (Bolt)

In some embodiments, the recombinant silk polypeptide is self-assembled into a semicrystalline state in which the crystalline portion is characterized by beta-sheet crosslinks that are resistant to solubility in water at pH from 3-8, other polar and non polar solvents (hexanol, hexane, benzene), oils, waxes, surfactants (anionic, non-ionic, cationic, amphoteric) but easily dispersed in these materials to form a heterogeneous dispersion.

In some embodiments, the recombinant silk polypeptide exists as a randomly structured gel. This gel can also include the presence of preservative and chelating agents. In various aspects, the recombinant silk polypeptide exists as a hollow powder. The hollow powder can be milled such that the powder incorporated into the composition as a milled powder. The powder can also include or be mixed with preservative and chelating agents.

In some embodiments, the recombinant silk polypeptide exists as a hollow powder suspended in an aqueous, polar, non-polar, oil, wax, or surfactant diluent. This mixture can also include the presence of preservative and chelating agents.

The recombinant silk polypeptide can be included in the composition in an amount of about 0.01 wt % to about 30 wt % based on the total weight of the composition. For example, the recombinant silk polypeptide can be included in the composition, based on the total weight of the composition, in an amount of about 0.05 wt % to about 5 wt %, about 0.5 wt % to about 5 wt %, about 0.01 wt % to about 0.5 wt %, about 0.1 wt % to about 0.5 wt %, about 0.05 wt % to about 5 wt %, about 0.5 wt % to about 5 wt %, about 5 wt % to about 20 wt %, about 5 wt % to about 30 wt %, about 25 wt % to about 30 wt %, about 10 wt % to about 25 wt %, or about 1 wt % to about 5 wt %.

In some embodiments, the recombinant silk polypeptide forms a distinctive and detectable film on the skin and hair when applied within a leave-on formulation or wash-off formulation.

In some embodiments, the recombinant silk polypeptide helps to cleanse or exfoliate when applied within a wash-off formula.

In some embodiments, the recombinant silk polypeptide can be detected within a formulation (leave-on or wash-off) as evidenced by visual inspection with microscopy where a powder can be observed at 5-100× objective. Additionally, the recombinant silk polypeptide can be detected by a high molecular weight peak between 50 kDa and 90 kDa using SEC-HPLC.

In some embodiments, the recombinant silk polypeptide is compatible (meaning its structure and performance is maintained) with a wide variety of common cosmetic components such as polar and non-polar solvents, oils, waxes, fatty acids, humectants, and (sunscreen) actives.

Due to the difference in recombinant silk polypeptide swelling in different solvents, these differences can be used as formulation processing aids. For example, recombinant silk polypeptide can be added to a formulation in the unswelled state within an oil, wax, or non-polar solvent, and then upon coming in contact with water will swell. The recombinant silk polypeptide is fully or partially removed from skin and hair by water and fully removed by surfactants.

### Definitions

The following terms, unless otherwise indicated, shall be understood to have the following meanings:

The term “stability”, as used herein with respect to silk proteins, refers to the ability of the product not to form a gelation, the ability of the product to resist degradation, and the ability to resist discoloration or turbidity that is due to the self-aggregation of silk proteins. For example, U.S. Patent Publication No. 2015/0079012 (Wray et al.) is directed to the use of humectant, including glycerol to increase the shelf-stability of skincare products comprising full-length silk fibroin. U.S. Pat. No. 9,187,538 is directed to a skincare formulation comprising full-length silk fibroin that is shelf stable for up to 10 days. Both of these publications are incorporated herein by reference in their entirety.

The term “polynucleotide” or “nucleic acid molecule” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native internucleoside bonds, or both. The nucleic acid can be in any topological conformation. For instance, the nucleic acid can be single-stranded, double-stranded, triple-stranded, quadruplexed, partially double-stranded, branched, hairpinned, circular, or in a padlocked conformation.

Unless otherwise indicated, and as an example for all sequences described herein under the general format “SEQ ID NO:”, “nucleic acid comprising SEQ ID NO:1” refers to a nucleic acid, at least a portion of which has either (i) the sequence of SEQ ID NO:1, or (ii) a sequence complementary to SEQ ID NO:1. The choice between the two is dictated by the context. For instance, if the nucleic acid is used as a probe, the choice between the two is dictated by the requirement that the probe be complementary to the desired target.

An “isolated” RNA, DNA or a mixed polymer is one which is substantially separated from other cellular components that naturally accompany the native polynucleotide in its natural host cell, e.g., ribosomes, polymerases and genomic sequences with which it is naturally associated.

An “isolated” organic molecule (e.g., a silk protein) is one which is substantially separated from the cellular components (membrane lipids, chromosomes, proteins) of the host cell from which it originated, or from the medium in which the host cell was cultured. The term does not require that the biomolecule has been separated from all other chemicals, although certain isolated biomolecules may be purified to near homogeneity.

The term “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids.

An endogenous nucleic acid sequence in the genome of an organism (or the encoded protein product of that sequence) is deemed “recombinant” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). By way of example, a promoter sequence can be substituted (e.g., by homologous recombination) for the native promoter of a gene in the genome of a host cell, such that this gene has an altered expression pattern. This gene would now become “recombinant” because it is separated from at least some of the sequences that naturally flank it.

A nucleic acid is also considered “recombinant” if it contains any modifications that do not naturally occur to the corresponding nucleic acid in a genome. For instance, an endogenous coding sequence is considered “recombinant” if it contains an insertion, deletion or a point mutation introduced artificially, e.g., by human intervention. A “recombinant nucleic acid” also includes a nucleic acid integrated into a host cell chromosome at a heterologous site and a nucleic acid construct present as an episome.

The term “peptide” as used herein refers to a short polypeptide, e.g., one that is typically less than about 50 amino acids long and more typically less than about 30 amino acids long. The term as used herein encompasses analogs and mimetics that mimic structural and thus biological function.

The term “polypeptide” encompasses both naturally occurring and non-naturally occurring proteins, and fragments, mutants, derivatives and analogs thereof. A polypeptide may be monomeric or polymeric. Further, a polypeptide may comprise a number of different domains each of which has one or more distinct activities.

The term “isolated protein” or “isolated polypeptide” is a protein or polypeptide that by virtue of its origin or source of derivation (1) is not associated with naturally associated components that accompany it in its native state, (2) exists in a purity not found in nature, where purity can be adjudged with respect to the presence of other cellular material (e.g., is free of other proteins from the same species) (3) is expressed by a cell from a different species, or (4) does not occur in nature (e.g., it is a fragment of a polypeptide found in nature or it includes amino acid analogs or derivatives not found in nature or linkages other than standard peptide bonds). Thus, a polypeptide that is chemically synthesized or synthesized in a cellular system different from the cell from which it naturally originates will be “isolated” from its naturally associated components. A polypeptide or protein may also be rendered substantially free of naturally associated components by isolation, using protein purification techniques well known in the art. As thus defined, “isolated” does not necessarily require that the protein, polypeptide, peptide or oligopeptide so described has been physically removed from its native environment.

The term “polypeptide fragment” refers to a polypeptide that has a deletion, e.g., an amino-terminal and/or carboxy-terminal deletion compared to a full-length polypeptide. In a preferred embodiment, the polypeptide fragment is a contiguous sequence in which the amino acid sequence of the fragment is identical to the corresponding positions in the naturally-occurring sequence. Fragments typically are at least 5, 6, 7, 8, 9 or 10 amino acids long, preferably at least 12, 14, 16 or 18 amino acids long, more preferably at least 20 amino acids long, more preferably at least 25, 30, 35, 40 or 45, amino acids, even more preferably at least 50 or 60 amino acids long, and even more preferably at least 70 amino acids long.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences.) As used herein, homology between two regions of amino acid sequence (especially with respect to predicted structural similarities) is interpreted as implying similarity in function.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art. See, e.g., Pearson, 1994, Methods Mol. Biol. 24:307-31 and 25:365-89 (herein incorporated by reference).

The twenty conventional amino acids and their abbreviations follow conventional usage. See Immunology—A Synthesis (Golub and Gren eds., Sinauer Associates, Sunderland, Mass., 2^{nd} ed. 1991), which is incorporated herein by reference. Stereoisomers (e.g., D-amino acids) of the twenty conventional amino acids, unnatural amino acids such as α-, α-disubstituted amino acids, N-alkyl amino acids, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, N-methylarginine, and other similar amino acids and imino acids (e.g., 4-hydroxyproline). In the polypeptide notation used herein, the left-hand end corresponds to the amino terminal end and the right-hand end corresponds to the carboxy-terminal end, in accordance with standard usage and convention.

The following six groups each contain amino acids that are conservative substitutions for one another: 1) Serine(S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for polypeptides, which is sometimes also referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using a measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild-type protein and a mutein thereof. See, e.g., GCG Version 6.1.

A useful algorithm when comparing a particular polypeptide sequence to a database containing a large number of sequences from different organisms is the computer program BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

Preferred parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

Preferred parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62. The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences. Pearson, Methods Enzymol. 183:63-98 (1990) (incorporated by reference herein). For example, percent sequence identity between amino acid sequences can be determined using FASTA with its default parameters (a word size of 2 and the PAM250 scoring matrix), as provided in GCG Version 6.1, herein incorporated by reference.

Throughout this specification and aspects, the word “comprise” or variations such as “comprises” or “comprising,” will be understood to imply the inclusion of a stated integer or group of integers but not the exclusion of any other integer or group of integers.

The term “glass transition” as used herein refers to the transition of a substance or composition from a hard, rigid or “glassy” state into a more pliable, “rubbery” or “viscous” state.

The term “glass transition temperature” as used herein refers to the temperature at which a substance or composition undergoes a glass transition.

The term “melt transition” as used herein refers to the transition of a substance or composition from a rubbery state to a less-ordered liquid phase.

The term “melting temperature” as used herein refers to the temperature range over which a substance undergoes a melt transition.

The term “plasticizer” as used herein refers to any molecule that interacts with a polypeptide sequence to prevent the polypeptide sequence from forming tertiary structures and bonds and/or increases the mobility of the polypeptide sequence.

The term “powder” as used herein refers to a composition that is present in granular form, which may or may not be complexed or agglomerated with a solvent such as water or serum. The term “dry powder” may be used interchangeably with the term “powder;” however, “dry powder” as used herein simply refers to the gross appearance of the granulated material and is not intended to mean that the material is completely free of complexed or agglomerated solvent unless otherwise indicated. Dry powder may be produced by spray-drying, lyophilization, and/or according to methods known in the art.

The term “carrier” refers to a recombinant protein used for surface hydration, surface cleansing, surface defense, surface detoxification, surface exfoliation, surface improvement, coloring, and/or delivery of various additives or solvents, including, but not limited to, water, glycerin, alcohols, siloxane, oils, humectants, emollients, occlusive agents, active agents, and/or cosmetic adjuvants to a surface like skin, hair, or nails. The carrier as used herein comprises an outer shell and hollow core, e.g., 18B protein.

The term “cosmetics” as used herein includes make-up, foundation, skin care, hair care, and nail care products.

The term “make-up” as used herein refers to products that leave color on the face, including foundation, blacks and browns, i.e., mascara, concealers, eye liners, brow colors, eye shadows, blushers, lip colors, powders, solid emulsion compact, and so forth.

The term “foundation” as used herein refers to liquid, cream, mousse, pancake, compact, concealer or like product created or reintroduced by cosmetic companies to even out the overall coloring of the skin.

The term “skin care products” as used herein refer to those used to treat or care for, or somehow moisturize, improve, or clean the skin. Products contemplated by the phrase “skin care products” include, but are not limited to, creams, mists, serums, cleansing gels, ampules, adhesives, patches, bandages, toothpaste, anhydrous occlusive moisturizers, antiperspirants, deodorants, personal cleansing products, powder laundry detergent, fabric softener towels, occlusive drug delivery patches, nail polish, powders, tissues, wipes, hair conditioners-anhydrous, shaving creams, and the like.

The term “sagging” as used herein means the laxity, slackness, or the like condition of skin that occurs as a result of loss of, damage to, alterations to, and/or abnormalities in dermal elastin, muscle and/or subcutaneous fat.

The terms “treating” or “treatment” as used herein refer to the treatment (e.g., alleviation or elimination of symptoms and/or cure) and/or prevention or inhibition of the condition (e.g., a skin condition) or relief of symptoms.

Exemplary methods and materials are described below, although methods and materials similar or equivalent to those described herein can also be used in the practice of the present invention and will be apparent to those of skill in the art. All publications and other references mentioned herein are incorporated by reference in their entirety. In case of conflict, the present specification, including definitions, will control. The materials, methods, and examples are illustrative only and not intended to be limiting.

### Recombinant Silk Proteins

The present disclosure describes embodiments of the invention including fibers synthesized from synthetic proteinaceous copolymers (i.e., recombinant polypeptides). Suitable proteinaceous co-polymers are discussed in U.S. Patent Publication No. 2016/0222174, published Aug. 45, 2016, U.S. Patent Publication No. 2018/0111970, published Apr. 26, 2018, and U.S. Patent Publication No. 2018/0057548, published Mar. 1, 2018, each of which are incorporated by reference herein in its entirety.

In some embodiments, the synthetic proteinaceous copolymers are made from silk-like polypeptide sequences. In some embodiments, the silk-like polypeptide sequences are 1) block copolymer polypeptide compositions generated by mixing and matching repeat domains derived from silk polypeptide sequences and/or 2) recombinant expression of block copolymer polypeptides having sufficiently large size (approximately 40 kDa) to form useful molded body compositions by secretion from an industrially scalable microorganism. Large (approximately 40 kDa to approximately 100 kDa) block copolymer polypeptides engineered from silk repeat domain fragments, including sequences from almost all published amino acid sequences of silk polypeptides, can be expressed in the modified microorganisms described herein. In some embodiments, silk polypeptide sequences are matched and designed to produce highly expressed and secreted polypeptides capable of molded body formation.

In some embodiments, block copolymers are engineered from a combinatorial mix of silk polypeptide domains across the silk polypeptide sequence space. In some embodiments, the block copolymers are made by expressing and secreting in scalable organisms (e.g., yeast, fungi, and gram positive bacteria). In some embodiments, the block copolymer polypeptide comprises 0 or more N-terminal domains (NTD), 1 or more repeat domains (REP), and 0 or more C-terminal domains (CTD). In some aspects of the embodiment, the block copolymer polypeptide is >100 amino acids of a single polypeptide chain. In some embodiments, the block copolymer polypeptide comprises a domain that is at least 80%, 81%, 82%, 83%, 84%, 85%, 86%, 87%, 88%, 89%, 90%, 91%, 92%, 93%, 94%, 95%, 96%, 97%, 98%, or 99% identical to a sequence of a block copolymer polypeptide as disclosed in International Publication No. WO/2015/042164, “Methods and Compositions for Synthesizing Improved Silk Fibers,” incorporated by reference in its entirety.

Several types of native spider silks have been identified. The mechanical properties of each natively spun silk type are believed to be closely connected to the molecular composition of that silk. See, e.g., Garb, J. E., et al., Untangling spider silk evolution with spidroin terminal domains, BMC Evol. Biol., 10:243 (2010); Bittencourt, D., et al., Protein families, natural history and biotechnological aspects of spider silk, Genet. Mol. Res., 11:3 (2012); Rising, A., et al., Spider silk proteins: recent advances in recombinant production, structure-function relationships and biomedical applications, Cell. Mol. Life Sci., 68:2, pg. 169-184 (2011); and Humenik, M., et al., Spider silk: understanding the structure-function relationship of a natural fiber, Prog. Mol. Biol. Transl. Sci., 103, pg. 131-85 (2011). For example:

Aciniform (AcSp) silks tend to have high toughness, a result of moderately high strength coupled with moderately high extensibility. AcSp silks are characterized by large block (“ensemble repeat”) sizes that often incorporate motifs of poly serine and GPX. Tubuliform (TuSp or Cylindrical) silks tend to have large diameters, with modest strength and high extensibility. TuSp silks are characterized by their poly serine and poly threonine content, and short tracts of poly alanine. Major Ampullate (MaSp) silks tend to have high strength and modest extensibility. MaSp silks can be one of two subtypes: MaSp1 and MaSp2. MaSp1 silks are generally less extensible than MaSp2 silks, and are characterized by poly alanine, GX, and GGX motifs. MaSp2 silks are characterized by poly alanine, GGX, and GPX motifs. Minor Ampullate (MiSp) silks tend to have modest strength and modest extensibility. MiSp silks are characterized by GGX, GA, and poly A motifs, and often contain spacer elements of approximately 100 amino acids. Flagelliform (Flag) silks tend to have very high extensibility and modest strength. Flag silks are usually characterized by GPG, GGX, and short spacer motifs.

The properties of each silk type can vary from species to species, and spiders leading distinct lifestyles (e.g. sedentary web spinners vs. vagabond hunters) or that are evolutionarily older may produce silks that differ in properties from the above descriptions (for descriptions of spider diversity and classification, see Hormiga, G., and Griswold, C. E., Systematics, phylogeny, and evolution of orb-weaving spiders, Annu. Rev. Entomol. 59, pg. 487-512 (2014); and Blackedge, T. A. et al., Reconstructing web evolution and spider diversification in the molecular era, Proc. Natl. Acad. Sci. U.S.A., 106:13, pg. 5229-5234 (2009)). However, synthetic block copolymer polypeptides having sequence similarity and/or amino acid composition similarity to the repeat domains of native silk proteins can be used to manufacture on commercial scales consistent molded bodies that have properties that recapitulate the properties of corresponding molded bodies made from natural silk polypeptides.

In some embodiments, a list of putative silk sequences can be compiled by searching GenBank for relevant terms, e.g. “spidroin” “fibroin” “MaSp”, and those sequences can be pooled with additional sequences obtained through independent sequencing efforts. Sequences are then translated into amino acids, filtered for duplicate entries, and manually split into domains (NTD, REP, CTD). In some embodiments, candidate amino acid sequences are reverse translated into a DNA sequence optimized for expression in Pichia (Komagataella) pastoris. The DNA sequences are each cloned into an expression vector and transformed into Pichia (Komagataella) pastoris. In some embodiments, various silk domains demonstrating successful expression and secretion are subsequently assembled in combinatorial fashion to build silk molecules capable of molded body formation.

Silk polypeptides are characteristically composed of a repeat domain (REP) flanked by non-repetitive regions (e.g., C-terminal and N-terminal domains). In an embodiment, both the C-terminal and N-terminal domains are between 75-350 amino acids in length. The repeat domain exhibits a hierarchical architecture. The repeat domain comprises a series of blocks (also called repeat units). The blocks are repeated, sometimes perfectly and sometimes imperfectly (making up a quasi-repeat domain), throughout the silk repeat domain. The length and composition of blocks varies among different silk types and across different species. Table 1A lists examples of block sequences from selected species and silk types, with further examples presented in Rising, A. et al., Spider silk proteins: recent advances in recombinant production, structure-function relationships and biomedical applications, Cell Mol. Life Sci., 68:2, pg 169-184 (2011); and Gatesy, J. et al., Extreme diversity, conservation, and convergence of spider silk fibroin sequences, Science, 291:5513, pg. 2603-2605 (2001). In some cases, blocks may be arranged in a regular pattern, forming larger macro-repeats that appear multiple times (usually 2-8) in the repeat domain of the silk sequence. Repeated blocks inside a repeat domain or macro-repeat, and repeated macro-repeats within the repeat domain, may be separated by spacing elements. In some embodiments, block sequences comprise a glycine rich region followed by a polyA region. In some embodiments, short (˜1-10) amino acid motifs appear multiple times inside of blocks. For the purpose of this invention, blocks from different natural silk polypeptides can be selected without reference to circular permutation (i.e., identified blocks that are otherwise similar between silk polypeptides may not align due to circular permutation). Thus, for example, a “block” of SGAGG (SEQ ID NO: 1) is, for the purposes of the present invention, the same as GSGAG (SEQ ID NO: 2) and the same as GGSGA (SEQ ID NO: 3); they are all just circular permutations of each other. The particular permutation selected for a given silk sequence can be dictated by convenience (usually starting with a G) more than anything else. Silk sequences obtained from the NCBI database can be partitioned into blocks and non-repetitive regions.

TABLE 1A
Samples of Block Sequences

| | Silk | Representative Block |
|---|---|---|
| Species | Type | Amino Acid Sequence |
| Aliatypus | Fibroin 1 | GAASSSSTIITTKSASASAAADASAAATASAASRS |
| gulosus | | SANAAASAFAQSFSSILLESGYFCSIFGSSISSSY |
| | | AAAIASAASRAAAESNGYTTHAYACAKAVASAVER |
| | | VTSGADAYAYAQAISDALSHALLYTGRLNTANANS |
| | | LASAFAYAFANAAAQASASSASAGAASASGAASAS |
| | | GAGSAS (SEQ ID NO: 4) |
| Plectreurys | Fibroin 1 | GAGAGAGAGAGAGAGAGSGASTSVSTSSSSGSGAG |
| tristis | | AGAGSGAGSGAGAGSGAGAGAGAGGAGAGFGSGLG |
| | | LGYGVGLSSAQAQAQAQAAAQAQAQAQAQAYAAAQ |
| | | AQAQAQAQAQAAAAAAAAAAA (SEQ ID NO: |
| | | 5) |
| Plectreurys | Fibroin 4 | GAAQKQPSGESSVATASAAATSVTSGGAPVGKPGV |
| tristis | | PAPIFYPQGPLQQGPAPGPSNVQPGTSQQGPIGGV |
| | | GGSNAFSSSFASALSLNRGFTEVISSASATAVASA |
| | | FQKGLAPYGTAFALSAASAAADAYNSIGSGANAFA |
| | | YAQAFARVLYPLVQQYGLSSSAKASAFASAIASSF |
| | | SSGTSGQGPSIGQQQPPVTISAASASAGASAAAVG |
| | | GGQVGQGPYGGQQQSTAASASAAAATATS (SEQ |
| | | ID NO: 6) |
| Araneus | TuSp | GNVGYQLGLKVANSLGLGNAQALASSLSQAVSAVG |
| gemmoides | | VGASSNAYANAVSNAVGQVLAGQGILNAANAGSLA |
| | | SSFASALSSSAASVASQSASQSQAASQSQAAASAF |
| | | RQAASQSASQSDSRAGSQSSTKTTSTSTSGSQADS |
| | | RSASSSASQASASAFAQQSSASLSSSSSFSSAFSS |
| | | ATSISAV (SEQ ID NO: 7) |
| Argiope | TuSp | GSLASSFASALSASAASVASSAAAQAASQSQAAAS |
| aurantia | | AFSRAASQSASQSAARSGAQSISTTTTTSTAGSQA |
| | | ASQSASSAASQASASSFARASSASLAASSSFSSAF |
| | | SSANSLSALGNVGYQLGFNVANNLGIGNAAGLGNA |
| | | LSQAVSSVGVGASSSTYANAVSNAVGQFLAGQGIL |
| | | NAANA (SEQ ID NO: 8) |
| Deinopis | TuSp | GASASAYASAISNAVGPYLYGLGLFNQANAASFAS |
| spinosa | | SFASAVSSAVASASASAASSAYAQSAAAQAQAASS |
| | | AFSQAAAQSAAAASAGASAGAGASAGAGAVAGAGA |
| | | VAGAGAVAGASAAAASQAAASSSASAVASAFAQSA |
| | | SYALASSSAFANAFASATSAGYLGSLAYQLGLTTA |
| | | YNLGLSNAQAFASTLSQAVTGVGL (SEQ ID |
| | | NO: 9) |
| Nephila | TuSp | GATAASYGNALSTAAAQFFATAGLLNAGNASALAS |
| clavipes | | SFARAFSASAESQSFAQSQAFQQASAFQQAASRSA |
| | | SQSAAEAGSTSSSTTTTTSAARSQAASQSASSSYS |
| | | SAFAQAASSSLATSSALSRAFSSVSSASAASSLAY |
| | | SIGLSAARSLGIADAAGLAGVLARAAGALGQ |
| | | (SEQ ID NO: 10) |
| Argiope | Flag | GGAPGGGPGGAGPGGAGFGPGGGAGFGPGGGAGFG |
| trifasciata | | PGGAAGGPGGPGGPGGPGGAGGYGPGGAGGYGPGG |
| | | VGPGGAGGYGPGGAGGYGPGGSGPGGAGPGGAGGE |
| | | GPVTVDVDVTVGPEGVGGGPGGAGPGGAGFGPGGG |
| | | AGFGPGGAPGAPGGPGGPGGPGGPGGPGGVGPGGA |
| | | GGYGPGGAGGVGPAGTGGFGPGGAGGFGPGGAGGF |
| | | GPGGAGGFGPAGAGGYGPGGVGPGGAGGFGPGGVG |
| | | PGGSGPGGAGGEGPVTVDVDVSV (SEQ ID NO: |
| | | 11) |
| Nephila | Flag | GVSYGPGGAGGPYGPGGPYGPGGEGPGGAGGPYGP |
| clavipes | | GGVGPGGSGPGGYGPGGAGPGGYGPGGSGPGGYGP |
| | | GGSGPGGYGPGGSGPGGYGPGGSGPGGYGPGGYGP |
| | | GGSGPGGSGPGGSGPGGYGPGGTGPGGSGPGGYGP |
| | | GGSGPGGSGPGGYGPGGSGPGGFGPGGSGPGGYGP |
| | | GGSGPGGAGPGGVGPGGFGPGGAGPGGAAPGGAGP |
| | | GGAGPGGAGPGGAGPGGAGPGGAGPGGAGGAGGAG |
| | | GSGGAGGSGGTTIIEDLDITIDGADGPITISEELP |
| | | ISGAGGSGPGGAGPGGVGPGGSGPGGVGPGGSGPG |
| | | GVGPGGSGPGGVGPGGAGGPYGPGGSGPGGAGGAG |
| | | GPGGAYGPGGSYGPGGSGGPGGAGGPYGPGGEGPG |
| | | GAGGPYGPGGAGGPYGPGGAGGPYGPGGEGGPYGP |
| | | (SEQ ID NO: 12) |
| Latrodectus | AcSp | GINVDSDIGSVTSLILSGSTLQMTIPAGGDDLSGG |
| hesperus | | YPGGFPAGAQPSGGAPVDFGGPSAGGDVAAKLARS |
| | | LASTLASSGVFRAAFNSRVSTPVAVQLTDALVQKI |
| | | ASNLGLDYATASKLRKASQAVSKVRMGSDTNAYAL |
| | | AISSALAEVLSSSGKVADANINQIAPQLASGIVLG |
| | | VSTTAPQFGVDLSSINVNLDISNVARNMQASIQGG |
| | | PAPITAEGPDFGAGYPGGAPTDLSGLDMGAPSDGS |
| | | RGGDATAKLLQALVPALLKSDVFRAIYKRGTRKQV |
| | | VQYVTNSALQQAASSLGLDASTISQLQTKATQALS |
| | | SVSADSDSTAYAKAFGLAIAQVLGTSGQVNDANVN |
| | | QIGAKLATGILRGSSAVAPRLGIDLS (SEQ ID |
| | | NO: 13) |
| Argiope | AcSp | GAGYTGPSGPSTGPSGYPGPLGGGAPFGQSGFGGS |
| trifasciata | | AGPQGGFGATGGASAGLISRVANALANTSTLRTVL |
| | | RTGVSQQIASSVVQRAAQSLASTLGVDGNNLARFA |
| | | VQAVSRLPAGSDTSAYAQAFSSALFNAGVLNASNI |
| | | DTLGSRVLSALLNGVSSAAQGLGINVDSGSVQSDI |
| | | SSSSSFLSTSSSSASYSQASASSTS (SEQ ID |
| | | NO: 14) |
| Uloborus | AcSp | GASAADIATAIAASVATSLQSNGVLTASNVSQLSN |
| diversus | | QLASYVSSGLSSTASSLGIQLGASLGAGFGASAGL |
| | | SASTDISSSVEATSASTLSSSASSTSVVSSINAQL |
| | | VPALAQTAVLNAAFSNINTQNAIRIAELLTQQVGR |
| | | QYGLSGSDVATASSQIRSALYSVQQGSASSAYVSA |
| | | IVGPLITALSSRGVVNASNSSQIASSLATAILQFT |
| | | ANVAPQFGISIPTSAVQSDLSTISQSLTAISSQTS |
| | | SSVDSSTSAFGGISGPSGPSPYGPQPSGPTFGPGP |
| | | SLSGLTGFTATFASSFKSTLASSTQFQLIAQSNLD |
| | | VQTRSSLISKVLINALSSLGISASVASSIAASSSQ |
| | | SLLSVSA (SEQ ID NO: 15) |
| Euprosthenops | MaSp1 | GGQGGQGQGRYGQGAGSSAAAAAAAAAAAAAA |
| australis | | (SEQ ID NO: 16) |
| Tetragnatha | MaSp1 | GGLGGGQGAGQGGQQGAGQGGYGSGLGGAGQGASA |
| kauaiensis | | AAAAAAA (SEQ ID NO: 17) |
| Argiope | MaSp2 | GGYGPGAGQQGPGSQGPGSGGQQGPGGLGPYGPSA |
| aurantia | | AAAAAAA (SEQ ID NO: 18) |
| Deinopis | MaSp2 | GPGGYGGPGQQGPGQGQYGPGTGQQGQGPSGQQGP |
| spinosa | | AGAAAAAAAAA (SEQ ID NO: 19) |
| Nephila | MaSp2 | GPGGYGLGQQGPGQQGPGQQGPAGYGPSGLSGPGG |
| clavata | | AAAAAAA (SEQ ID NO: 20) |
| Deinopis | MiSp | GAGYGAGAGAGGGAGAGTGYGGGAGYGTGSGAGYG |
| Spinosa | | AGVGYGAGAGAGGGAGAGAGGGTGAGAGGGAGAGY |
| | | GAGTGYGAGAGAGGGAGAGAGAGAGAGAGAGSGAG |
| | | AGYGAGAGYGAGAGAGGVAGAGAAGGAGAAGGAGA |
| | | AGGAGAAGGAGAGAGAGSGAGAGAGGGARAGAGG |
| | | (SEQ ID NO: 21) |
| Latrodectus | MiSp | GGGYGRGQGAGAGVGAGAGAAAGAAAIARAGGYGQ |
| hesperus | | GAGGYGQGQGAGAAAGAAAGAGAGGYGQGAGGYGR |
| | | GQGAGAGAGAGAGARGYGQGAGAGAAAGAAASAGA |
| | | GGYGQGAGGYGQGQGAGAAAGAAASAGAGGYGQGA |
| | | GGYGQGQGA (SEQ ID NO: 22) |
| Nephila | MiSp | GAGAGGAGYGRGAGAGAGAAAGAGAGAAAGAGAGA |
| clavipes | | GGYGGQGGYGAGAGAGAAAAAGAGAGGAAGYSRGG |
| | | RAGAAGAGAGAAAGAGAGAGGYGGQGGYGAGAGAG |
| | | AAAAAGAGSGGAGGYGRGAGAGAAAGAGAAAGAGA |
| | | GAGGYGGQGGYGAGAGAAAAA (SEQ ID NO: |
| | | 23) |
| Nephilengys | MiSp | GAGAGVGGAGGYGSGAGAGAGAGAGAASGAAAGAA |
| cruentata | | AGAGAGGAGGYGTGQGYGAGAGAGAGAGAGGAGGY |
| | | GRGAGAGAGAGAGGAGGYGAGQGYGAGAGAGAAAA |
| | | AGDGAGAGGAGGYGRGAGAGAGAGAAAGAGAGGAG |
| | | GYGAGQGYGAGAGAGAAAGAGAGGAGGYGAGQGYG |
| | | AGAGAGAAAAA (SEQ ID NO: 24) |
| Uloborus | MiSp | GSGAGAGSGYGAGAGAGAGSGYGAGSSASAGSAIN |
| diversus | | TQTVTSSTTTSSQSSAAATGAGYGTGAGTGASAGA |
| | | AASGAGAGYGGQAGYGQGAGASARAAGSGYGAGAG |
| | | AAAAAGSGYGAGAGAGAGSGYGAGAAA (SEQ ID |
| | | NO: 25) |
| Uloborus | MiSp | GAGAGYRGQAGYIQGAGASAGAAAAGAGVGYGGQA |
| diversus | | GYGQGAGASAGAAAAAGAGAGRQAGYGQGAGASAG |
| | | AAAAGAGAGRQAGYGQGAGASAGAAAAGADAGYGG |
| | | QAGYGQGAGASAGAAASGAGAGYGGQAGYGQGAGA |
| | | SAGAAAAGAGAGYLGQAGYGQGAGASAGAAAGAGA |
| | | GYGGQAGYGQGTGAAASAAASSA (SEQ ID NO: |
| | | 26) |
| Araneus | MaSp1 | GGQGGQGGYGGLGSQGAGQGGYGAGQGAAAAAAAA |
| ventricosus | | GGAGGAGRGGLGAGGAGQGYGAGLGGQGGAGQAAA |
| | | AAAAGGAGGARQGGLGAGGAGQGYGAGLGGQGGAG |
| | | QGGAAAAAAAAGGQGGQGGYGGLGSQGAGQGGYGA |
| | | GQGGAAAAAAAAGGQGGQGGYGGLGSQGAGQGGYG |
| | | GRQGGAGAAAAAAAA (SEQ ID NO: 27) |
| Dolomedes | MaSp1 | GGAGAGQGSYGGQGGYGQGGAGAATATAAAAGGAG |
| tenebrosus | | SGQGGYGGQGGLGGYGQGAGAGAAAAAAAAAGGAG |
| | | AGQGGYGGQGGQGGYGQGAGAGAAAAAAGGAGAGQ |
| | | GGYGGQGGYGQGGGAGAAAAAAAASGGSGSGQGGY |
| | | GGQGGLGGYGQGAGAGAGAAASAAAA (SEQ ID |
| | | NO: 28) |
| Nephilengys | MaSp | GGAGQGGYGGLGGQGAGAAAAAAGGAGQGGYGGQG |
| cruentata | | AGQGAAAAAASGAGQGGYEGPGAGQGAGAAAAAAG |
| | | GAGQGGYGGLGGQGAGQGAGAAAAAAGGAGQGGYG |
| | | GLGGQGAGQGAGAAAAAAGGAGQGGYGGQGAGQGA |
| | | AAAAAGGAGQGGYGGLGSGQGGYGRQGAGAAAAAA |
| | | AA (SEQ ID NO: 29) |
| Nephilengys | MaSp | GGAGQGGYGGLGGQGAGAAAAAAGGAGQGGYGGQG |
| cruentata | | AGQGAAAAAASGAGQGGYGGPGAGQGAGAAAAAAG |
| | | GAGQGGYGGLGGQGAGQGAGAAAAAAGGAGQGGYG |
| | | GQGAGQGAAAAAAGGAGQGGYGGLGSGQGGYGGQG |
| | | AGAAAAAGGAGQGGYGGLGGQGAGQGAGAAAAAA |
| | | (SEQ ID NO: 30) |

Fiber-forming block copolymer polypeptides from the blocks and/or macro-repeat domains, according to certain embodiments of the invention, is described in International Publication No. WO/2015/042164, incorporated by reference. Natural silk sequences obtained from a protein database such as GenBank or through de novo sequencing are broken up by domain (N-terminal domain, repeat domain, and C-terminal domain). The N-terminal domain and C-terminal domain sequences selected for the purpose of synthesis and assembly into fibers or molded bodies include natural amino acid sequence information and other modifications described herein. The repeat domain is decomposed into repeat sequences containing representative blocks, usually 1-8 depending upon the type of silk, which capture critical amino acid information while reducing the size of the DNA encoding the amino acids into a readily synthesizable fragment. In some embodiments, a properly formed block copolymer polypeptide comprises at least one repeat domain comprising at least 1 repeat sequence, and is optionally flanked by an N-terminal domain and/or a C-terminal domain.

In some embodiments, a repeat domain comprises at least one repeat sequence. In some embodiments, the repeat sequence is 150-300 amino acid residues. In some embodiments, the repeat sequence comprises a plurality of blocks. In some embodiments, the repeat sequence comprises a plurality of macro-repeats. In some embodiments, a block or a macro-repeat is split across multiple repeat sequences.

In some embodiments, the repeat sequence starts with a glycine, and cannot end with phenylalanine (F), tyrosine (Y), tryptophan (W), cysteine (C), histidine (H), asparagine (N), methionine (M), or aspartic acid (D) to satisfy DNA assembly requirements. In some embodiments, some of the repeat sequences can be altered as compared to native sequences. In some embodiments, the repeat sequences can be altered such as by addition of a serine to the C terminus of the polypeptide (to avoid terminating in F, Y, W, C, H, N, M, or D). In some embodiments, the repeat sequence can be modified by filling in an incomplete block with homologous sequence from another block. In some embodiments, the repeat sequence can be modified by rearranging the order of blocks or macrorepeats.

In some embodiments, non-repetitive N- and C-terminal domains can be selected for synthesis. In some embodiments, N-terminal domains can be by removal of the leading signal sequence, e.g., as identified by SignalP (Peterson, T. N., et. Al., SignalP 4.0: discriminating signal peptides from transmembrane regions, Nat. Methods, 8:10, pg. 785-786 (2011).

In some embodiments, the N-terminal domain, repeat sequence, or C-terminal domain sequences can be derived from Agelenopsis aperta, Aliatypus gulosus, Aphonopelma seemanni, Aptostichus sp. AS217, Aptostichus sp. AS220, Araneus diadematus, Araneus gemmoides, Araneus ventricosus, Argiope amoena, Argiope argentata, Argiope bruennichi, Argiope trifasciata, Atypoides riversi, Avicularia juruensis, Bothriocyrtum californicum, Deinopis Spinosa, Diguetia canities, Dolomedes tenebrosus, Euagrus chisoseus, Euprosthenops australis, Gasteracantha mammosa, Hypochilus thorelli, Kukulcania hibernalis, Latrodectus hesperus, Megahexura fulva, Metepeira grandiosa, Nephila antipodiana, Nephila clavata, Nephila clavipes, Nephila madagascariensis, Nephila pilipes, Nephilengys cruentata, Parawixia bistriata, Peucetia viridans, Plectreurys tristis, Poecilotheria regalis, Tetragnatha kauaiensis, or Uloborus diversus.

In some embodiments, the silk polypeptide nucleotide coding sequence can be operatively linked to an alpha mating factor nucleotide coding sequence. In some embodiments, the silk polypeptide nucleotide coding sequence can be operatively linked to another endogenous or heterologous secretion signal coding sequence. In some embodiments, the silk polypeptide nucleotide coding sequence can be operatively linked to a 3×FLAG nucleotide coding sequence. In some embodiments, the silk polypeptide nucleotide coding sequence is operatively linked to other affinity tags such as 6-8 His residues.

In some embodiments, the recombinant silk polypeptides are based on recombinant spider silk protein fragment sequences derived from MaSp2, such as from the species Argiope bruennichi. In some embodiments, the synthesized fiber contains protein molecules that include two to twenty repeat units, in which a molecular weight of each repeat unit is greater than about 20 kDa. Within each repeat unit of the copolymer are more than about 60 amino acid residues, often in the range 60 to 100 amino acids that are organized into a number of “quasi-repeat units.” In some embodiments, the repeat unit of a polypeptide described in this disclosure has at least 95% sequence identity to a MaSp2 dragline silk protein sequence.

The repeat unit of the proteinaceous block copolymer that forms fibers with good mechanical properties can be synthesized using a portion of a silk polypeptide. These polypeptide repeat units contain alanine-rich regions and glycine-rich regions, and are 150 amino acids in length or longer. Some exemplary sequences that can be used as repeats in the proteinaceous block copolymers of this disclosure are provided in in co-owned PCT Publication WO 2015/042164, incorporated by reference in its entirety, and were demonstrated to express using a Pichia expression system.

In some embodiments, the silk protein comprises: at least two occurrences of a repeat unit, the repeat unit comprising: more than 150 amino acid residues and having a molecular weight of at least 10 kDa; an alanine-rich region with 6 or more consecutive amino acids, comprising an alanine content of at least 80%; a glycine-rich region with 12 or more consecutive amino acids, comprising a glycine content of at least 40% and an alanine content of less than 30%; and wherein the fiber comprises at least one property selected from the group consisting of a modulus of elasticity greater than 550 cN/tex, an extensibility of at least 10% and an ultimate tensile strength of at least 15 cN/tex.

In some embodiments, wherein the recombinant silk protein comprises repeat units wherein each repeat unit has at least 95% sequence identity to a sequence that comprises from 2 to 20 quasi-repeat units; each quasi-repeat unit comprises {GGY-[GPG-X1] n1-GPS-(A) n2} (SEQ ID NO: 35), wherein for each quasi-repeat unit; X1 is independently selected from the group consisting of SGGQQ (SEQ ID NO: 31), GAGQQ (SEQ ID NO: 32), GQGOPY (SEQ ID NO: 33), AGQQ (SEQ ID NO: 34), and SQ; and n1 is from 4 to 8, and n2 is from 6-10. The repeat unit is composed of multiple quasi-repeat units.

In some embodiments, 3 “long” quasi repeats are followed by 3 “short” quasi-repeat units. As mentioned above, short quasi-repeat units are those in which n1=4 or 5. Long quasi-repeat units are defined as those in which n1-6, 7 or 8. In some embodiments, all of the short quasi-repeats have the same X1 motifs in the same positions within each quasi-repeat unit of a repeat unit. In some embodiments, no more than 3 quasi-repeat units out of 6 share the same X1 motifs.

In additional embodiments, a repeat unit is composed of quasi-repeat units that do not use the same X1 more than two occurrences in a row within a repeat unit. In additional embodiments, a repeat unit is composed of quasi-repeat units where at least 1, 2, 3, 4, 5, 6, 7, 8, 9, 10, 11, 12, 13, 14, 15, 16, 17, 18, 19 or 20 of the quasi-repeats do not use the same X1 more than 2 times in a single quasi-repeat unit of the repeat unit.

In some embodiments, the recombinant silk polypeptide comprises the polypeptide sequence of SEQ ID NO: 36 (i.e., 18B). In some embodiments, the repeat unit is a polypeptide comprising SEQ ID NO: 37. These sequences are provided in Table 1B:

TABLE 1B
Exemplary polypeptides sequences of recombinant
protein and repeat unit

| SEQ | |
|---|---|
| ID | Polypeptide Sequence |
| SEQ | GGYGPGAGQQGPGSGGQQGPGGQGPYGSGQQGPGGAGQQGPGGQGPYGPGAAAAAAAAA |
| ID | GYGPGAGQQGPGGAGQQGPGSQGPGGQGPYGPGAGQQGPGSQGPGSGGQQGPGGQGPYS |
| NO: | AAAAAAAAAGGYGPGAGQRSQGPGGQGPYGPGAGQQGPGSQGPGSGGQQGPGGQGPYGS |
| 36 | AAAAAAAAGGYGPGAGQQGPGSQGPGSGGQQGPGGQGPYGPGAAAAAAAVGGYGPGAGQ |
| | QGPGSQGPGSGGQQGPGGQGPYGPSAAAAAAAAGGYGPGAGQQGPGSQGPGSGGQQGPG |
| | QGPYGPSAAAAAAAAGGYGPGAGQQGPGSGGQQGPGGQGPYGSGQQGPGGAGQQGPGGQ |
| | PYGPGAAAAAAAAAGGYGPGAGQQGPGGAGQQGPGSQGPGGQGPYGPGAGQQGPGSQGP |
| | SGGQQGPGGQGPYGPSAAAAAAAAAGGYGPGAGQRSQGPGGQGPYGPGAGQQGPGSQGP |
| | SGGQQGPGGQGPYGPSAAAAAAAAGGYGPGAGQQGPGSQGPGSGGQQGPGGQGPYGPGA |
| | AAAAAVGGYGPGAGQQGPGSQGPGSGGQQGPGGQGPYGPSAAAAAAAAGGYGPGAGQQG |
| | GSQGPGSGGQQGPGGQGPYGPSAAAAAAAAGGYGPGAGQQGPGSGGQQGPGGQGPYGSG |
| | QGPGGAGQQGPGGQGPYGPGAAAAAAAAAGGYGPGAGQQGPGGAGQQGPGSQGPGGQGP |
| | GPGAGQQGPGSQGPGSGGQQGPGGQGPYGPSAAAAAAAAAGGYGPGAGQRSQGPGGQGP |
| | GPGAGQQGPGSQGPGSGGQQGPGGQGPYGPSAAAAAAAAGGYGPGAGQQGPGSQGPGSG |
| | QQGPGGQGPYGPGAAAAAAAVGGYGPGAGQQGPGSQGPGSGGQQGPGGQGPYGPSAAAA |
| | AAAGGYGPGAGQQGPGSQGPGSGGQQGPGGQGPYGPSAAAAAAAA |
| SEQ | GGYGPGAGQQGPGSGGQQGPGGQGPYGSGQQGPGGAGQQGPGGQGPYGPGAAAAAAAAA |
| ID | GYGPGAGQQGPGGAGQQGPGSQGPGGQGPYGPGAGQQGPGSQGPGSGGQQGPGGQGPYS |
| NO: | AAAAAAAAAGGYGPGAGQRSQGPGGQGPYGPGAGQQGPGSQGPGSGGQQGPGGQGPYGS |
| 37 | AAAAAAAAGGYGPGAGQQGPGSQGPGSGGQQGPGGQGPYGPGAAAAAAAVGGYGPGAGQ |
| | QGPGSQGPGSGGQQGPGGQGPYGPSAAAAAAAAGGYGPGAGQQGPGSQGPGSGGQQGPG |
| | QGPYGPSAAAAAAAA |

In some embodiments, the structure of fibers formed from the described recombinant silk polypeptides form beta-sheet structures, beta-turn structures, or alpha-helix structures. In some embodiments, the secondary, tertiary and quaternary protein structures of the formed fibers are described as having nanocrystalline beta-sheet regions, amorphous beta-turn regions, amorphous alpha helix regions, randomly spatially distributed nanocrystalline regions embedded in a non-crystalline matrix, or randomly oriented nanocrystalline regions embedded in a non-crystalline matrix. Without intending to be limited by theory, the structural properties of the proteins within the spider silk are theorized to be related to fiber mechanical properties. Crystalline regions in a fiber have been linked with the tensile strength of a fiber, while the amorphous regions have been linked to the extensibility of a fiber. The major ampullate (MA) silks tend to have higher strengths and less extensibility than the flagelliform silks, and likewise the MA silks have higher volume fraction of crystalline regions compared with flagelliform silks. Furthermore, theoretical models based on the molecular dynamics of crystalline and amorphous regions of spider silk proteins, support the assertion that the crystalline regions have been linked with the tensile strength of a fiber, while the amorphous regions have been linked to the extensibility of a fiber. Additionally, the theoretical modeling supports the importance of the secondary, tertiary and quaternary structure on the mechanical properties of recombinant protein fibers (RPFs). For instance, both the assembly of nano-crystal domains in a random, parallel and serial spatial distributions, and the strength of the interaction forces between entangled chains within the amorphous regions, and between the amorphous regions and the nano-crystalline regions, influenced the theoretical mechanical properties of the resulting fibers.

In some embodiments, the molecular weight of the silk protein may range from 20 kDa to 2000 kDa, or greater than 20 kDa, or greater than 10 kDa, or greater than 5 kDa, or from 5 to 400 kDa, or from 5 to 300 kDa, or from 5 to 200 kDa, or from 5 to 100 kDa, or from 5 to 50 kDa, or from 5 to 500 kDa, or from 5 to 1000 kDa, or from 5 to 2000 kDa, or from 10 to 400 kDa, or from 10 to 300 kDa, or from 10 to 200 kDa, or from 10 to 100 kDa, or from 10 to 50 kDa, or from 10 to 500 kDa, or from 10 to 1000 kDa, or from 10 to 2000 kDa, or from 20 to 400 kDa, or from 20 to 300 kDa, or from 20 to 200 kDa, or from 40 to 300 kDa, or from 40 to 500 kDa, or from 20 to 100 kDa, or from 20 to 50 kDa, or from 20 to 500 kDa, or from 20 to 1000 kDa, or from 20 to 2000 kDa.

### Characterization of Recombinant Spider Silk Polypeptide Powder Impurities and Degradation

Different recombinant spider silk polypeptides have different physiochemical properties such as melting temperature and glass transition temperature based on the strength and stability of the secondary and tertiary structures formed by the proteins. Silk polypeptides form beta sheet structures in a monomeric form. In the presence of other monomers, the silk polypeptides form a three-dimensional crystalline lattice of beta sheet structures. The beta sheet structures are separated from, and interspersed with, amorphous regions of polypeptide sequences.

Beta sheet structures are extremely stable at high temperatures—the melting temperature of beta-sheets is approximately 257° C. as measured by fast scanning calorimetry. See Cebe et al., Beating the Heat—Fast Scanning Melts Silk Beta Sheet Crystals, Nature Scientific Reports 3:1130 (2013). As beta sheet structures are thought to stay intact above the glass transition temperature of silk polypeptides, it has been postulated that the structural transitions seen at the glass transition temperature of recombinant silk polypeptides are due to increased mobility of the amorphous regions between the beta sheets.

Plasticizers lower the glass transition temperature and the melting temperature of silk proteins by increasing the mobility of the amorphous regions and potentially disrupting beta sheet formation. Suitable plasticizers used for this purpose include, but are not limited to, water and polyalcohols (polyols) such as glycerol, triglycerol, hexaglycerol, and decaglycerol. Other suitable plasticizers include, but are not limited to, Dimethyl Isosorbite; adiptic acid; amide of dimethylaminopropyl amine and caprylic/capric acid; acetamide; and any combination thereof.

As hydrophilic portions of silk polypeptides can bind ambient water present in the air as humidity, water will almost always be present, the bound ambient water may plasticize silk polypeptides. In some embodiments, a suitable plasticizer may be glycerol, present either alone or in combination with water or other plasticizers. Other suitable plasticizers are discussed above.

In addition, in instances where recombinant silk polypeptides are produced by fermentation and recovered as recombinant silk polypeptide powder from the same, there may be impurities present in the recombinant silk polypeptide powder that act as plasticizers or otherwise inhibit the formation of tertiary structures. For example, residual lipids and sugars may act as plasticizers and thus influence the glass transition temperature of the protein by interfering with the formation of tertiary structures.

Various well-established methods may be used to assess the purity and relative composition of recombinant silk polypeptide powder or composition. Size Exclusion Chromatography separates molecules based on their relative size and can be used to analyze the relative amounts of recombinant silk polypeptide in its full-length polymeric and monomeric forms as well as the amount of high, low and intermediate molecular weight impurities in the recombinant silk polypeptide powder. Similarly, Rapid High Performance Liquid Chromatography may be used to measure various compounds present in a solution such as monomeric forms of the recombinant silk polypeptide. Ion Exchange Liquid Chromatography may be used to assess the concentrations of various trace molecules in solution, including impurities such as lipids and sugars. Other methods of chromatography and quantification of various molecules such as mass spectrometry are well established in the art.

Depending on the embodiment, the recombinant silk polypeptide may have a purity calculated based on the amount of the recombinant silk polypeptide in its monomeric form by weight relative to the other components of the recombinant silk polypeptide powder. In various instances, the purity can range from 50% by weight to 90% by weight, depending on the type of recombinant silk polypeptide and the techniques used to recover, separate and post-process the recombinant silk polypeptide powder.

Both Size Exclusion Chromatography and Reverse Phase High Performance Liquid Chromatography are useful in measuring full-length recombinant silk polypeptide, which makes them useful techniques for determining whether processing steps have degraded the recombinant silk polypeptide by comparing the amount of full-length silk polypeptide in a composition before and after processing. In various embodiments of the present invention, the amount of full-length recombinant silk polypeptide present in a composition before and after processing may be subject to minimal degradation. The amount of degradation may be in the range 0.001% by weight to 10% by weight, or 0.01% by weight to 6% by weight, e.g. less than 10% or 8% or 6% by weight, or less than 5% by weight, less than 3% by weight or less than 1% by weight.

### Recombinant Silk Containing Component

The recombinant silk containing component includes the recombinant silk polypeptide. The recombinant silk containing component can consists of the recombinant silk polypeptide. The recombinant silk containing component can include the recombinant silk polypeptide with a solvent and/or one or more additives, such as preservatives and chelating agents. The recombinant silk containing component can include the recombinant silk polypeptide in an amount, based on the total weight of the recombinant silk containing component, of about 1 wt % to about 40 wt % or in any other suitable amount needed to achieve a final desired loading the recombinant silk polypeptide in the cosmetic, skin or hair care composition.

Without intending to be limited by theory, in various embodiments of the present invention, inducing the recombinant silk containing component may be used in applications where it is desirable to prevent the aggregation of the monomeric recombinant silk polypeptide into its crystalline polymeric form or to control the transition of the recombinant silk polypeptide into its crystalline polymeric form at a later stage in processing. In other embodiments, such inducing is not required.

The amount of degradation of the recombinant silk polypeptide may be measured using various techniques. As discussed above, the amount of degradation of the recombinant silk polypeptide may be measured using Size Exclusion Chromatography to measure the amount of full-length recombinant silk polypeptide present. In various embodiments, the recombinant silk polypeptide is degraded in an amount of less than 6.0 weight % after it is formed into a molded body. In another embodiment, the recombinant silk polypeptide is degraded in an amount of less than 4.0 weight % after molding, less than 3.0 weight %, less than 2.0 weight %, or less than 1.0 weight %, such that the amount of degradation may be in the range 0.001% by weight to 10%, 8%, 6%, 4%, 3%, 2% or 1% by weight, or 0.01% by weight to 6%, 4%, 3%, 2% or 1% by weight. In another embodiment, the recombinant silk protein in the composition is substantially non-degraded. In a similar embodiment, the recombinant silk protein in the composition is substantially non-degraded over a period of time, at least 1 day, 1 month, 1 year, or 5 years.

In some embodiments, the component comprising the recombinant silk protein is physically stable. In various embodiments, the component remains in its material form, e.g., a powder, for a prolonged period of time, with a prolonged shelf life. On prolonged use, the recombinant silk containing component remains substantially stable.

The 18B protein is more stable in a dried form than in an aqueous slurry. In some embodiments, spray-dried recombinant silk is obtained as follows: a slurry composition comprising extracted recombinant silk is kept chilled during the drying step. It is pumped to a tall form spray dryer where the moisture content of the resulting powder is tightly controlled. As the protein powder is hydroscopic, the final powder collection and packout is performed to minimize reintroduction of moisture. The design of the packaging material should minimize moisture and light exposure.

In some embodiments, recovery and separation of the recombinant silk polypeptide from a cell culture is performed as follows: i) extraction and separation, ii) urea removal by ultrafiltration, iii) washing by precipitation, iv) salt removal and protein concentration, and v) spray drying.

In some embodiments, to freeze-dry a composition it is cooled until it solidifies and placed under reduced pressure to cause the most volatile ingredients in the composition to sublime. The solid residue may form a single mass which requires milling to form a fine powder. A typical freeze-dried powder comprises porous irregular shaped particles and readily hydrates. As freeze-drying does not require strong heat it is used to produce powders which comprise volatile ingredients. In some embodiments, the recombinant silk containing component is deep freeze-dried at a temperature below about −100° C.

After formation of the recombinant silk containing component, the crystallinity of the recombinant silk containing component can increase, thereby strengthening the composition. In some embodiments, the recombinant silk containing component stays the same or decreases. In some embodiments, the crystallinity index of the recombinant silk containing component as measured by X-ray crystallography is from 2% to 90%. In some other embodiments, the crystallinity index of the recombinant silk containing component as measured by X-ray crystallography is at least 3%, at least 4%, at least 5%, at least 6%, or at least 7%.

In some embodiments of the present invention, the recombinant silk containing component is a solid or film. In some embodiments, the recombinant silk containing component is a powder. In some embodiments, the solid or film will be substantially homogeneous meaning that the material, as inspected by light microscopy, has a low amount or does not have any inclusions or precipitates. In some embodiments, light microscopy may be used to measure birefringence which can be used as a proxy for alignment of the recombinant silk into a three-dimensional lattice. Birefringence is the optical property of a material having a refractive index that depends on the polarization and propagation of light. Specifically, a high degree of axial order as measured by birefringence can be linked to high tensile strength. In some embodiments, recombinant silk solids and films will have minimal birefringence. In various embodiments, the solid is a bead. In some other embodiments, the solid functions as an exfoliant. The recombinant silk solid may be in the form of a gentle skin scrub for the skin. In some embodiments, the material form is a roll, pellet, sheet, or flake.

In some embodiments, the recombinant silk protein comprises a hollow core and/or a shell. In some embodiments, the recombinant silk protein ranges from about 1 I·im to about 30 I·im in diameter, about 5 I·im to about 20 I·im, or about 10 I·im to about 50 I·im in diameter, while recombinant silk protein in water ranges from about 20 to about 80 I·im in diameter, about 30 I·im to about 70 I·im, or about 40 I·im to about 100 I·im in diameter. Prior to incorporate into the compositions of the disclosure, the recombinant silk protein hollow powder can be milled and incorporated as a milled powder.

### Solvents

In some embodiments, the recombinant silk containing component can include one or more solvents. For example, the recombinant silk polypeptide can be suspended in a solvent. The solvent can be an aqueous solvent, an alcohol, or an oil-based solvent. For example, the solvent can be one or more of water, glycerin, deionized water, olive oil, and pentylene glycol. For example, the recombinant silk polypeptide can be treated with a solvent such that the hollow core contains the solvent such as liquid water or glycerin, either in form of liquid water itself, or as a liquid aqueous solution, as an emulsion containing liquid water, or as an aqueous dispersion. In some embodiments, the recombinant silk containing component comprises about a 25 wt % solution in glycerin.

In some embodiments, the solvent is water. Without intending to be limited by theory, subjecting the recombinant silk polypeptide to a solvent such as water results in a recombinant silk polypeptide that has expanded or swelled, wherein the protein functions as a carrier containing the solvent (e.g., water). These compositions can be stored dry and partially rehydratable after immersion in water to directly form a liquid or semi-liquid aqueous suspension of expanded particles.

In some embodiments, the recombinant silk protein may expand a portion of the hollow core. In some other embodiments, the recombinant silk protein may expand a portion of the shell. In such embodiments where the solvent is water, the recombinant silk protein transforms into a hydrogel. In other embodiments where the solvent is water, the recombinant silk protein transforms into a paste. In various embodiments, heat and/or pressure may be added to further process the recombinant silk protein compositions.

In some embodiments, a solvent is generally present in a proportion ranging from 55 to 90% by weight relative to the total weight of the recombinant silk polypeptide. This range includes all specific values and subranges there between, including 60%, 65%, 70%, 75%, 80%, and 85% by weight. In some embodiments, the recombinant silk protein is insoluble in various solvents, including water at various different pH levels, glycerin, alcohols, siloxane, and oils.

In some embodiments, the solvent is an aqueous type. In such embodiments, the solvent is water. The solvent may have a pH ranging from 6 to 12. In some embodiments, the solvent has a pH of 6. In some other embodiments, the solvent has a pH ranging from 0 to 5, from 2 to 7, from 4 to 9, from 6 to 11, from 8 to 13, or from 10 to 14.

In other embodiments, the solvent includes a mixture of various volatile organic solvents, in order to obtain relatively short drying times. In some embodiments, the solvent is an alcohol.

Solvents may include water, ethyl alcohol, toluene, methylene chloride, isopropanol, n-butyl alcohol, castor oil, organopolysiloxane oils, ethylene glycol monoethyl ether, diethylene glycol monobutyl ether, diethylene glycol monoethyl ether, dimethyl sulphoxide, dimethyl formamide and tetrahydrofuran.

In some embodiments, the organopolysiloxane oil may be volatile, non-volatile, or a mixture of volatile and non-volatile silicones. The term “non-volatile” as used in this context refers to those silicones that are liquid under ambient conditions and have a flash point (under one atmospheric of pressure) of or greater than about 100° C. The term “volatile” as used in this context refers to all other silicone oils. Suitable organopolysiloxanes can be selected from a wide variety of silicones spanning a broad range of volatilities and viscosities. Suitable silicones are disclosed in U.S. Pat. No. 5,069,897, issued Dec. 3, 1991, which is incorporated by reference herein in its entirety. Examples of suitable organopolysiloxanes include, but are not limited to, polyalkylsiloxanes, alkyl substituted dimethicones, dimethiconols, polyalkylaryl siloxanes, and mixtures thereof. For instance, polyalkylsiloxanes, dimethicones and cyclomethicones may be used.

In some embodiments, the solvent is a vegetable oil and hydrogenated vegetable oil. In some embodiments, the solvent is a free fatty acid. Examples of vegetable oils and hydrogenated vegetable oils include safflower oil, castor oil, coconut oil, cottonseed oil, menhaden oil, palm kernel oil, palm oil, peanut oil, soybean oil, rapeseed oil, linseed oil, rice bran oil, pine oil, sesame oil, sunflower seed oil, partially and fully hydrogenated oils from the foregoing sources, and mixtures thereof. Animal fats and oils, e.g., cod liver oil, lanolin and derivatives thereof such as acetylated lanolin and isopropyl lanolate, may be used. Also useful are C4-C20 alkyl ethers of polypropylene glycols, C1-C20 carboxylic acid esters of polypropylene glycols, and di-C8-C30 alkyl ethers, examples of which include PPG-14 butyl ether, PPG-15 stearyl ether, dioctyl ether, dodecyl octyl ether, and mixtures thereof.

The compositions of the present invention may be substantially free of semi-solid hydrocarbons such as petrolatum, lanolin and lanolin derivatives, sterols (e.g., ethoxylated soya sterols), high molecular weight polybutenes and cocoa butter. By “substantially free,” as used herein, means that the concentration of the semi-solid hydrocarbons is less than 10%, or less than 5% or less than 2% or 0%.

### Recombinant Silk Proteins as a Cosmetics Formulation

In various embodiments, the recombinant silk protein is compounded into a silk cosmetic or skincare product (e.g., solutions applied to the skin or hair). Specifically, the recombinant silk protein can be incorporated into a recombinant silk containing component to be used as a base for a cosmetic or skincare product where the recombinant silk polypeptide is present in the base in its monomeric or less-crystalline form. In some embodiments, the recombinant silk containing component may be used as a base for a cosmetic or skincare product where the recombinant silk polypeptide is present in the base in a semi-crystalline form. In such embodiments, the recombinant silk polypeptide is not present in the base in its monomeric form.

For example, recombinant silk proteins can be used in compositions for stabilization of one or more actives. Recombinant silk proteins can be included in formulations to aid or provide controlled release of actives and/or to reduce redness and/or irritation from such actives. Such actives can include, but are not limited to, retinol, vitamin C, and resveratrol.

Recombinant silk proteins can be used in sunscreen applications, for example, with mineral SPFs and can aid in reducing the white cast resulting from mineral SPFs.

Recombinant silk proteins can be used with natural oils and emulsifies. For example, they can be blended, for example, for cold emulsification.

Recombinant silk proteins can be used as ingredient replacements, such as silicone elastomer replacements, to provide a greener composition.

Recombinant silk proteins can be used in compositions with or as pigments.

Compositions can include recombinant silk proteins in combination with poly-quaternary compound, such as poly-quaternary amines, or other charge molecules for wet/dry combing.

Compositions can include recombination silk proteins that can be present in the composition to provide or enhance one or more properties such as encapsulation or one or more components, controlled release properties, stabilizing properties, silicone replacement, and/or film forming properties.

For example, combinations can include the recombinant silk protein with peptides, such as keratin, or other actives, whereby the recombinant silk protein functions as an encapsulant.

For example, the recombinant silk protein can be included in combination with cleansing acids, such as hyaluronic acid, to provide skin barrier protection/wound healing properties.

For example, the recombinant silk protein can be provided in compositions with ceramides and/or in deodorant and/or antiperspirant compositions as a silicone replacement to provide silicone free compositions or substantially silicon free compositions.

Recombinant silk proteins can be included in compositions with anti-acne actives and provide encapsulant, stabilizer, and/or controlled release properties to the composition.

Recombinant silk proteins can be included in combination with fragrances to provide a fragrance composition with encapsulant, stabilizer, and/or controlled release properties.

Recombinant silk proteins can be included in sunscreen compositions with chemical UV filters and/or mineral UV filters to provide sunscreens that are substantially silicone free, as well as impart film forming properties and reduce white cast resulting from mineral UV filters.

Recombinant silk properties can be provided in insect repellents as an encapsulant and/or film former.

In fluoride-containing oral care compositions, the recombinant silk protein can be used as an encapsulant, provide and/or aid in providing controlled release, and/or serve as a silicone replacement to make the composition substantially silicone free.

Recombinant silk proteins can be used in dry shampoo to bind and strengthen hair.

Recombinant silk proteins can be used as an encapsulant, stabilizer, and/or hair protector in hair dyes and/or bleaching compositions.

Recombinant silk proteins can be used in hair loss compositions, with actives, such as but not limited to, caffeine, minoxidil, finasteride, dutasteride, and provide controlled release and/or film forming properties.

Recombinant silk proteins can be used in a variety of compositions, including, but not limited to compositions for acne/blackhead removal, anti-hair loss, anti-aging, baby skin care, body care/cleansing, cleansing/moisturizing, cosmetic powders and mousses, deodorants/antiperspirants, exfoliating, eye cosmetics, facial care/cleansing, foundation and bb creams, fragrances, hair color, hair conditioning/treatment, hair styling, hand sanitizer, hand/foot care, lip cosmetics, male grooming, nail cosmetics, oral care, self-tanning, shampoo, skin treatment, skin whitening, soaps/cleansing bars, and/or sun care. Compositions for household care, oral care, and as insect repellants are also contemplated herein. Household care compositions can be, for example, home/furniture polishes, dishwashing detergents, laundry detergents, and fabric softeners. For example, the recombinant silk protein can be included as an anti-foaming agent.

The recombinant silk protein can have various properties and used as or with various components in cosmetic and personal care formulations, including, but not limited to, as absorbent, antibacterial, antioxidant, chelating agent, cleansing agent, coloring agent/pigment, emollient, emulsifier, exfoliant, film-forming agent, fragrance: synthetic and natural, humectant, irritant, occlusive/opacifying agent, peptides, ph adjuster/stabilizer, plant extracts, polymer, prebiotic/probiotic/postbiotic, preservative, Silicone, Solvent, and/or Suspending/Dispersing Agent, Texture Enhancer.

Recombinant silk proteins can be incorporated into various composition types. For example, recombinant silk proteins can be included in Pressed powders [eyes, cheeks, face], Loose powders [eyes, cheeks, face], Lipsticks, Lash/Brow Serum, Mascara, Lip glosses/treatments, Shampoos, Conditioners, Leave-in hair treatments, Vit A/CBD encapsulation, Sheet Mask, Cleansers, and/or SPF compositions.

Recombinant silk proteins can be useful, for example, in a color delivery system for vegan colorants that delivers more vibrantly than alt systems. For example, recombinant silk protein can be useful as a keratin alternative for hair rebuilding. For example, recombinant silk protein can be useful as a silicone alternative for softness. For example, recombinant silk protein can be useful as acrylate alternative for thickening. for example, recombinant silk protein can be useful as active delivery vehicle (encapsulation). for example, recombinant silk protein can be useful as uv/blue light/ir defense (physical defense and antioxidation. for example, recombinant silk protein can be useful as physical cleanser (e.g., microbead alternative). for example, recombinant silk protein can be useful as talc/mica alternative for absorbency/mattification/pigment filler.

In most embodiments, the cosmetic formulations are physically stable. In such embodiments, the recombinant silk protein and any other ingredients remain in its formulation for a prolonged period of time, with a prolonged shelf life. On prolonged use, the recombinant silk containing component remains substantially stable and the ingredients do not precipitate out of the formulation.

The composition of the invention may be used to apply the silk protein to the skin, nails, hair or mucous membranes, by contacting the composition with the skin, nails, hair or mucous membranes of a subject. The composition can be used with human subjects.

In most embodiments, the cosmetic formulations are non-toxic or non-allergenic to subject hosts to which the cosmetic is applied. It is also desirable in the art to produce cosmetic compositions for hair and epidermal contact which will not permanently stain tissue and which can be removed by ordinary washing with aqueous detergents.

The solids, films, emulsions, hydrogels, and other material forms discussed in various embodiments may contain various humectants, emollients, occlusive agents, active agents, and cosmetic adjuvants, depending on the embodiment and the desired efficacy of the formulation. In some embodiments, the recombinant silk protein functions as a carrier. In some embodiments, the recombinant silk protein is a carrier, delivering one or more agents to a surface such as skin, hair, or nails.

In some embodiments, the cosmetic formulation comprises a plasticizer. Suitable concentrations of plasticizer by weight in the composition ranges from, e.g., 1 to 60% by weight, 10 to 60% by weight, 10 to 50% by weight, 10 to 40% by weight, 15 to 40% by weight, 10 to 30% by weight, or 15 to 30% by weight. In some embodiments, the plasticizer is glycerol. In some embodiments, the plasticizer is triethanolamine, trimethylene glycol, polyethylene glycol, propylene glycol, sorbitol, sucrose, a saturated fatty acid, or an unsaturated fatty acid.

In the instance where water is used as a plasticizer, a suitable concentration of water by weight in the composition ranges from, e.g., 5 to 80% by weight, 15 to 70% by weight, 20 to 60% by weight, 25 to 50% by weight, 19 to 43% by weight, or 19 to 27% by weight. Where water is used in combination with another plasticizer, it may be present in a range of, e.g., 5 to 50% by weight, 15 to 43% by weight or 19 to 27% by weight.

In some embodiments, suitable plasticizers may include polyols (e.g., glycerol), water, lactic acid, ascorbic acid, phosphoric acid, ethylene glycol, propylene glycol, triethanolamine, acid acetate, propane-1,3-diol or any combination thereof. In various embodiments, the amount of plasticizer can vary according to the purity and relative composition of the recombinant silk protein. For example, a higher purity powder may have less impurities such as a low molecular weight compound that may act as a plasticizer and therefore require the addition of a higher percentage by weight of plasticizer.

In some embodiments, the composition comprises a humectant or emollient. The term “humectant” as used herein refers to a hygroscopic substance that forms a bond with water molecules. Suitable humectants include, but are not limited to glycerol, propylene glycol, polyethylene glycol, pentalyene glycol, tremella extract, sorbitol, dicyanamide, sodium lactate, hyaluronic acid, aloe vera extract, alpha-hydroxy acid and pyrrolidonecarboxylate (NaPCA).

The term “emollient” as used herein refers to a compound that provide skin a soft or supple appearance by filling in cracks in the skin surface. Suitable emollients include, but are not limited to shea butter, cocao butter, squalene, squalane, octyl octanoate, sesame oil, grape seed oil, natural oils containing oleic acid (e.g., sweet almond oil, argan oil, olive oil, avocado oil), natural oils containing gamma linoleic acid (e.g., evening primrose oil, borage oil), natural oils containing linoleic acid (e.g., safflower oil, sunflower oil), or any combination thereof.

In some instances, an emollient or humectant may be an occlusive agent, and the disclosure contemplates inclusion of an occlusive agent into the composition in various embodiments. The term “occlusive agent” refers to a compound that forms a barrier on the skin surface to retain moisture. Other suitable occlusive agents may include, but are not limited to beeswax, canuba wax, ceramides, vegetable waxes, lecithin, allantoin. Without intending to be limited by theory, the film-forming capabilities of the recombinant silk containing component presented herein make an occlusive agent that forms a moisture retaining barrier because the recombinant silk polypeptides act attract water molecules and also act as humectants.

Optionally, the cosmetic formulation comprises an active agent. The term “active agent” refers to any compound that has a known beneficial effect in a hair care, skincare, or cosmetic formulation, including pigment in cosmetic formulations. Various active agents include, but are not limited to, acetic acid (i.e., vitamin C), alpha hydroxyl acids, beta hydroxyl acids, zinc oxide, titanium dioxide, retinol, niacinamide, other recombinant proteins (either as full length sequences or hydrolyzed into subsequences or “peptides”), copper peptides, curcuminoids, glycolic acid, hydroquinone, kojic acid, l-ascorbic acid, alpha lipoic acid, azelaic acid, lactic acid, ferulic acid, mandelic acid, dimethylaminoethanol (DMAE), resveratrol, natural extracts containing antioxidants (e.g. green tea extract, pine tree extract), caffeine, alpha arbutin, coenzyme Q-10, and salicylic acid.

The term “cosmetic adjuvant” refers to various other agents used to create a cosmetic product with commercially desirable properties, including, without limitation, surfactants, emulsifiers, preserving agents and thickeners.

As described herein, in various embodiments, the recombinant silk protein may form a semi-solid or gel-like structure that is dispersible. In various embodiments where the recombinant silk protein is compounded into a skin care formulation, the recombinant silk protein may form a non-reversible three-dimensional structure such as a gel or film that transforms into a dispersible liquid upon the surface of the skin.

In various embodiments, the recombinant silk protein may be suspended in water (“aqueous suspended protein”) to form a recombinant silk containing component in the form of a film, gel, or base that can be incorporated (i.e., compounded) in a cosmetic or skincare formulation. Depending on the embodiment, the amount of recombinant silk protein to water in the aqueous suspended protein can vary, as can the relative ratio of recombinant silk polypeptide powder to additive in the recombinant silk protein. In some embodiments, the recombinant silk containing component will comprise 10-33% recombinant silk polypeptide powder by weight. In some embodiments, a different solvent than water will be used. In some embodiments, the recombinant silk protein is suspended in water to create an aqueous suspended protein that is 1-40% recombinant silk protein and 60-99% water. In a specific embodiment, the recombinant silk containing component is suspended in water to create an aqueous suspended protein that is 10% recombinant silk polypeptide powder by weight, 30% additive by weight and 60% water by weight based on the total weight of the recombinant silk containing component. In a specific embodiment, the protein is suspended in water to create an aqueous suspended protein that is 6% recombinant silk polypeptide powder by weight, 18% additive by weight and 76% water by weight based on the total weight of the recombinant silk containing component. In a specific embodiment, the protein is suspended in water to create an aqueous suspended protein that is 10% recombinant silk polypeptide powder by weight and 90% water by weight based on the total weight of the recombinant silk containing component.

Depending on the embodiment, the aqueous suspended protein may be optionally heated and agitated when it is re-suspended in water. In some embodiments, heating and agitating the aqueous suspended protein may result in a phase transformation of the recombinant silk polypeptides in the aqueous suspended protein. Specifically, heating and agitating the aqueous suspended protein results in three distinct phases that are assessed by centrifugation: 1) a gel phase that is distinct from the supernatant after centrifugation; 2) a colloidal phase that can be filtered from the supernatant after centrifugation; and 3) a solution phase that remains after filtering the colloidal phase from the supernatant. Various combinations of heat, agitation and centrifugation may be used, provided that the aqueous suspended protein must not be subject to prolonged heat in order to prevent degradation of the recombinant silk polypeptides. In a specific embodiment, the protein is subjected to gentle agitation at 90° C. for 5 minutes and centrifuged at 16,000 RCF for 30 minutes.

In various embodiments, either the various phases of the aqueous suspended protein (i.e., colloidal phase, gel phase and solution) or the aqueous suspended protein may be incorporated in a cosmetic or skincare formulation to provide a source of recombinant silk protein. Depending on the embodiment, the aqueous suspended protein may be subject to agitation with or without heat before incorporating into a skincare formulation. Optionally, the aqueous suspended protein may be separated in the above-discussed phases by centrifugation and/or filtering. Depending on the embodiment, the skincare formulation may be an emulsion (e.g., a cream or serum) or a primarily aqueous solution (e.g., a gel). In certain embodiments, the recombinant silk protein may be incorporated into any of the cosmetic, skin care, or hair care formulations described herein without aqueous resuspension. In these compositions, a homogenizer or similar equipment may be used to ensure that the recombinant silk protein is uniformly distributed in the composition.

In some embodiments, the aqueous suspended protein may be subject to heat and agitation, then cast onto a flat surface and dried into a film. In some embodiments, the aqueous suspended protein may be cast onto a flat surface and dried into a film without being subjected to heat and/or agitation. In such embodiments, the aqueous suspended protein may be cast onto a flat surface and dried into a film without being subjected to additional processing. In some embodiments, the aqueous suspended protein may be incorporated into an emulsion, then cast onto a flat surface and dried into a film. Depending on the embodiment, various different drying conditions may be used. Suitable drying conditions include drying at 60° C. or at 80° C. with and without a vacuum. In embodiments that use a vacuum, 15 Hg is a suitable amount of vacuum. Other methods of drying are well established in the art.

In various embodiments, the films comprising the aqueous suspended protein alone have a low melting temperature. In various embodiments, the films comprising the aqueous suspended protein alone have melting temperature that is less than body temperature (around 34-36° C.) and melts upon contact with skin. Without intending to be limited by theory, the recombinant silk polypeptide forms enough intermolecular interactions to make a semi-solid structure (i.e., film); however, this structure is reversible upon skin contact and can be re-formed after dispersion on the skin surface. In various embodiments, the film will have reduced crystallinity compared to the recombinant silk protein or the recombinant silk powder, as measured by Fourier-transform infrared spectroscopy (FTIR). In various embodiments, the films comprising the aqueous suspended protein do not melt upon contact with skin. In such embodiments, the film functions as a barrier. In various embodiments, the film is a hydrophobic film of low density. The film or barrier may range from about 1 I·im to about 50 I·im in thickness, from about 10 I·im to about 30 I·im, or from about 20 I·im to about 40 I·im in thickness. Upon contact with skin, the barrier may be formed on the surface of the epidermal layer, materializing a robust, non-specific adherence is made to the skin surface. In some embodiments, the thickness of the film changes depending on the concentration of recombinant silk protein and surface area of application.

In some embodiments, the barrier is long-lasting and prevents against one or more environmental stressors, including wind, humidity, harsh additives, pollution, abrasion, dirt, and grease. The barrier may withstand abrasion equivalent to at least 100 rubs by hand, at least 200 rubs, at least 400 rubs, at least 600 rubs, or at least 800 rubs.

In one specific embodiment, the aqueous suspended protein or the protein may be incorporated (e.g., homogenized) into an emulsion, then cast on a flat surface and lyophilized to create a porous film. Depending on the embodiment, various techniques may be used for lyophilization, including freezing the film at −80° C. for 30 minutes. Other lyophilization techniques will be well known to those skilled in the art.

In various embodiments, the above-described films can be used as a topical skincare agent. This film may be applied directly to the skin and can be re-hydrated to form a dispersible viscous substance that is incorporated into the skin. As discussed herein, various emollients, humectants, active agents, and other cosmetic adjuvants may be incorporated into the film. This film may be applied directly to the skin and adsorb to the skin due to contact with the skin, or after gently rubbing the film into the skin. In some embodiments, the film may be applied directly the skin and adsorb to the skin without additional rubbing or contact. In some embodiments, the protein resuspended in an aqueous solution may be applied to the face and then exposed to a coagulant such as propylene glycol via mist to form a gellable mask.

Depending on the embodiment, the film that is cast may be a flat film (i.e., with no surface variability) or may be cast on a mold that incorporates microstructures. In a specific embodiment, the film that is cast on a mold that incorporates microneedle structures to prick the surface of the skin and assist in delivery of active agents.

In an alternate embodiment, the aqueous suspended protein may be added to an emulsion that is used as a cosmetic product. The emulsion may be applied to skin or hair and then allowed to form a film on the surface of the skin upon drying. As discussed herein, various emollients, humectants, active agents, and other cosmetic adjuvants may be incorporated into the emulsion.

In some embodiments, the compositions of the disclosure may be liquid or semisolid, such as creams, lotions, and gels. The compositions useful in the subject invention may be made into a wide variety of product forms that are known in the art. These include, but are not limited to, powders, lotions, creams, gels, patches, serums, ampules, powders, sticks, sprays, ointments, pastes, mousses, ointments, liquids, emulsions, foams, or aerosols. These product forms may comprise several types of additives, as further discussed herein, including, but not limited to, solutions, aerosols, emulsions, gels, solids, and liposomes. The compounds which are active in the compositions and methods of this invention may be delivered topically by any means known to those of skill in the art.

In some other embodiments, the compositions may be basic cosmetic compositions such as facial cleansers, such as toilet water, cream, essence, cleansing foam and cleansing water; pack and body oil; color cosmetic compositions such as foundation, lipstick, mascara, and make-up base; hair product compositions such as shampoo, rinse, hair conditioner and hair gel; soap; and the like. The cosmetic formulation can be prepared in any method known in the art, using the recombinant silk containing component described herein, optionally together with at least one carrier and/or additive, which are commonly used in the field of preparing cosmetic compositions.

In some embodiments, the compositions comprise at least one cosmetic agent. Examples of cosmetic agents include emollients, humectants, colorants, pigments, fragrances, moisturizers, viscosity modifiers and any other cosmetic forming agent. One or more cosmetic agents can be included in the cosmetic composition. In another embodiment, additional active ingredients as known in the art and described herein may also be used, including, but not limited to, a skin softener, a skin permeation enhancer, a colorant, an aromatic, an emulsifier, and a thickener. Also, the cosmetic composition may further comprise a perfumery, a pigment, a bactericidal agent, an antioxidant, a preservative, and/or a moisturizer, as well as inorganic salts and synthetic polymer substances, for, e.g., the purpose of improving physical properties.

The composition may also be delivered topically via a lotion. Single emulsion skin care preparations, such as lotions and creams, of the oil-in-water type and water-in-oil type are well-known in the cosmetic art and are useful in the subject invention. Multiphase emulsion compositions, such as the water-in-oil-in-water type, are also useful in the subject invention. In general, such single or multiphase emulsions contain water, emollients, and emulsifiers as essential ingredients.

The compositions of the present invention can also be formulated into a solid formulation (e.g., a wax-based stick, soap bar composition, powder, bead, exfoliant, or a wipe containing liquid or powder).

The compositions of this invention can be formulated as a gel (e.g., an aqueous gel using a suitable gelling agent(s)). Suitable gelling agents for aqueous gels include, but are not limited to, natural gums, acrylic acid and acrylate polymers and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically comprise between about 0.1% and 5%, by weight, of such gelling agents. In some embodiments, such compositions include a combination of recombinant silk protein, water (Aqua), sodium C14-16 olefin sulfonate, glycerin, cocoa betaine, sodium benzoate, sodium hydroxide, calcium gluconate, sodium hyaluronate, propanediol, xanthan gum, gluconolactone, and tetrasodium glutamate diacetate. In some embodiments, compositions comprise a cleansing detergent, soap, serum, or toner. In a specific embodiment, the serum is aqueous-based. In another specific embodiment, the toner is alcohol-based.

The compositions useful in the present invention may be formulated as emulsions. If the composition is an emulsion, in some embodiments, from about 1% to about 10% or from about 2% to about 5% of the composition comprises an emulsifier. Emulsifiers may be nonionic, anionic or cationic. Suitable emulsifiers are disclosed in, for example, INCI Handbook, pp. 1673-1686. Lotions and creams can be formulated as emulsions. In some embodiments, the composition is an emulsion and the recombinant silk protein is an emulsifier. In some embodiments, the composition is an emulsion, the recombinant silk protein is an emulsifier, and the composition is free of other emulsifiers.

Yet another type of composition may be an ointment. An ointment may comprise a simple base of animal or vegetable oils or semi-solid hydrocarbons. An ointment may comprise from about 2% to about 10% of an emollient in addition to from about 0.1% to about 2% of a thickening agent. Examples of thickening agents include, e.g., cellulose derivatives (methyl cellulose and hydroxyl propylmethylcellulose), synthetic high molecular weight polymers (e.g., carboxyvinyl polymer and polyvinyl alcohol), plant hydrocolloids (e.g., karaya gum and tragacanth gum), clay thickeners (e.g., colloidal magnesium aluminum silicate and bentonite), carboxyvinyl polymers, carboxylic acid polymers, crosslinked polyacrylates, polyacrylamides, xanthan gum, and mixtures thereof.

The compositions useful in the subject invention may contain, in addition to the aforementioned components, a wide variety of additional oil-soluble materials and/or water-soluble materials conventionally used in compositions for use on skin, hair, and nails at their art-established levels.

The compositions of the present invention may be directly applied to the skin or may be applied onto other delivery implements such as wipes, sponges, brushes, and the like. The compositions may be used in products designed to be left on the skin, wiped from the skin, or rinsed off of the skin.

In some embodiments, the composition improves the appearance of skin, such as increasing skin firmness/plumpness, increasing elasticity, improving overall skin health, increasing hydration, accelerating and/or improving wound healing, improving pollution defense, reducing dermatological aging, decreasing skin fragility, preventing and reversing loss of collagen and/or elastin, preventing skin atrophy, promoting/accelerating cell turnover, increasing genetic expression, improving skin texture, preventing and decreasing fine lines and wrinkles, improving skin tone, enhancing skin thickness, decreasing pore size, minimizing skin discoloration, restoring skin luster, minimizing signs of fatigue, improving skin barrier function, minimizing skin dryness, preventing, reducing, or treating hyperpigmentation, improving the mitochondrial function of the skin, improves exfoliation, reduces toxicity, mattifying skin, reducing oxidative stress levels, attenuating pollution induced oxidative stress, attenuating UVA or UVB induced oxidative stress, or any combination thereof.

The compositions of various embodiments defend against pollutants and other irritants. As a result, many skin conditions, such as acne, the redness associated with rosacea (adult acne), and other inflammatory conditions can be actively managed by application of the cosmetic formulations.

### Other Additives

In some embodiments, a composition in accordance with the disclosure and/or the silicone replacement component thereof can include one or more additives. This can change the properties of the composition as it interacts with the skin. In some embodiments, the silk-based composition is submerged in the additive. In some embodiments, the composition/component is exposed to the additive mist or vapor. In one embodiment, an aqueous protein composition comprises or is submerged with or mixed with the additive. In some embodiments, a silk-based solid or semi-solid, such as a film, is submerged in or exposed to a vapor comprising the additive. In some embodiments, the silk-based gel is exposed to the additive prior to hallow powder formation (e.g., the silk-based gel and additive are co-spray dried together).

The additive can itself be inert or it can possess dermatological benefits of its own. The additive should also be physically and chemically compatible with the essential components described herein, and should not unduly impair stability, efficacy or other use benefits associated with the compositions of the present invention. The type of additive utilized in the present invention depends on the type of product form desired for the composition. In some embodiments, the additive is an acid textile dye.

Pigments are frequently added to cosmetic formulations to achieve a desired color for application to the skin. Such pigments are known and the concentrations required to achieve a desired coloring are readily determinable. Pigments may be inorganic or organic. Inorganic pigments include iron oxides (red, black, brown colors), manganese violet, ultramarines (green, blue, pink, red, or violet aluminum sulfosilicates), aquamarines, copper powder, mica, clays, silica, and titanium dioxide. Organic dyes that have been certified by the US FDA for cosmetic use generally have the prefix “D&C” and a suffix of a color and a number (for example, D&C Green #3).

Certain embodiments of the present invention contain from about 0% to about 30%, from about 1% to about 20%, from about 2% to about 15%, or from about 5% to about 15% of a colorant, on an anhydrous pigment weight basis. These are usually aluminum, barium or calcium salts or lakes. Dyes may be present at a concentration of from about 0% to about 3% and pearlizing agents and the like from 0% to about 10%. Such dyes in combination with recombinant silk proteins are stable and have a long shelf-life. The shelf-life of such compositions may be about 6 months, about 1 year, or about 2 years. In some embodiments, the shelf-life of such compositions may be at least 5 years.

There are no specific limitations as to the pigment, colorant, or filler powders used in the composition. Each may be a body pigment, inorganic white pigment, inorganic colored pigment, pearling agent, and the like. Specific examples are talc, mica, magnesium carbonate, calcium carbonate, magnesium silicate, aluminum magnesium silicate, silica, titanium dioxide, zinc oxide, red iron oxide, yellow iron oxide, black iron oxide, ultramarine, polyethylene powder, methacrylate powder, polystyrene powder, silk powder, crystalline cellulose, starch, titanated mica, iron oxide titanated mica, bismuth oxychloride, and the like.

Additional pigment/powder fillers include, but are not limited to, inorganic powders such as gums, chalk, Fuller's earth, kaolin, sericite, muscovite, phlogopite, synthetic mica, lepidolite, biotite, lithia mica, vermiculite, aluminum silicate, starch, smectite clays, alkyl and/or trialkyl aryl ammonium smectites, chemically modified magnesium aluminum silicate, organically modified montmorillonite clay, hydrated aluminum silicate, fumed aluminum starch octenyl succinate barium silicate, calcium silicate, magnesium silicate, strontium silicate, metal tungstate, magnesium, silica alumina, zeolite, barium sulfate, calcined calcium sulfate (calcined gypsum), calcium phosphate, fluorine apatite, hydroxyapatite, ceramic powder, metallic soap (zinc stearate, magnesium stearate, zinc myristate, calcium palmitate, and aluminum stearate), colloidal silicone dioxide, and boron nitride; organic powder such as polyamide resin powder (nylon powder), cyclodextrin, methyl polymethacrylate powder, copolymer powder of styrene and acrylic acid, benzoguanamine resin powder, poly(ethylene tetrafluoride) powder, and carboxyvinyl polymer, cellulose powder such as hydroxyethyl cellulose and sodium carboxymethyl cellulose, ethylene glycol monostearate; and inorganic white pigments such as magnesium oxide. Other useful powders are disclosed in U.S. Pat. No. 5,688,831, to El-Nokaly et al., issued Nov. 18, 1997, herein incorporated by reference in its entirety. These pigments and powders can be used independently or in combination.

Besides the silk protein, the composition according to the invention can further comprise a film-forming substance. Examples of film-forming substances include, e.g., cellulose derivatives, nitrocellulose, acrylic polymers or copolymers, acrylic, styrene, acrylate-styrene and vinyl resins, vinyl copolymers, polyester polymers, arylsulphonamide resins, and alkyde resins.

In some embodiments, the composition may include an amphoteric surfactant, a phospholipid, or a wax.

Examples of other additives include, but are not limited to, cannabidiol, foaming surfactants, depigmentation agents, reflectants, detangling/wet combing agents, amino acids and their derivatives, antimicrobial agents, allergy inhibitors, anti-acne agents, anti-aging agents, anti-wrinkling agents antiseptics, analgesics, antitussives, antipruritics, local anesthetics, anti-hair loss agents, hair growth promoting agents, hair growth inhibitor agents, antihistamines, antiinfectives, inflammation inhibitors, anti-emetics, anticholinergics, vasoconstrictors, vasodilators, wound healing promoters, peptides, polypeptides and proteins, deodorants and antiperspirants, medicament agents, skin emollients and skin moisturizers, skin firming agents, hair conditioners, hair softeners, hair moisturizers, vitamins, tanning agents, skin lightening agents, antifungals, depilating agents, shaving preparations, external analgesics, perfumes, counterirritants, hemorrhoidals, insecticides, poison ivy products, poison oak products, burn products, anti-diaper rash agents, prickly heat agents, make-up preparations, vitamins, herbal extracts, retinoids, flavenoids, sensates, anti-oxidants, skin conditioners, hair lighteners, chelating agents, cell turnover enhancers, sunscreens, anti-edema agents, collagen enhancers, and mixtures thereof.

Examples of suitable vitamins nonexclusively include vitamin B complex, including thiamine, nicotinic acid, biotin, pantothenic acid, choline, riboflavin, vitamin B6, vitamin B12, pyridoxine, inositol, camitine; vitamins A, C, D, E, K and their derivatives such as vitamin A palmitate and pro-vitamins, (e.g., panthenol (pro vitamin B5) and panthenol triacetate) and mixtures thereof.

Examples of sunscreen agents include, but are not limited to, avobenzone, benzophenones, bornelone, butyl paba, cinnamidopropyl trimethyl ammonium chloride, disodium distyrylbiphenyl disulfonate, paba, potassium methoxycinnamate, butyl methoxydibenzoylmethane, octyl methoxycinnamate, oxybenzone, octocrylene, octyl salicylate, phenylbenzimidazole sulfonic acid, ethyl hydroxypropyl aminobenzoate, menthyl anthranilate, aminobenzoic acid, cinoxate, diethanolamine methoxycinnamate, glyceryl aminobenzoate, titanium dioxide, zinc oxide, oxybenzone, Padimate O, red petrolatum, and mixtures thereof.

The amount of additive to be combined with the composition may vary depending upon, for example, the ability of the additive to penetrate through the skin, hair, or nail; the specific additive chosen; the particular benefit desired; the sensitivity of the user to the additive; the health condition, age, and skin, hair, and/or nail condition of the user; and the like. In sum, the additive is used in a “safe and effective amount,” which is an amount that is high enough to deliver a desired skin, hair, or nail benefit or to modify a certain condition to be treated, but is low enough to avoid serious side effects, at a reasonable risk to benefit ratio within the scope of sound medical judgment.

The invention illustratively disclosed herein suitably may be practiced in the absence of any component, ingredient, or step which is not specifically disclosed herein. Several examples are set forth below to further illustrate the nature of the invention and the manner of carrying it out. However, the invention should not be considered as being limited to the details thereof.

The compositions and methods of the present invention provide for skin equal or better performance for softness, quick absorption, easy spreadability (or “playtime”), lightweight film formation, and non-greasy afterfell as compared to compositions containing silicone elastomers. Additionally, if the skin is being treated with an SPF composition, then the invention provides equal or better performance for low white cast. The compositions and methods of the present invention provide for hair equal or better performance for long-lasting wear, shine, non-greasiness, frizz control, adding thickness to the hair, styling retention, electrostatic properties, resistance to heat, and UV-radiation and pollution defense.

### Examples

### Blurring Primer

w/Either 2% Silicones (Dimethicone) or 1% Bsilk

Ingredients

| | |
|---|---|
| Water | Cocoa Butter |
| Glycerin | Carbomer |
| Dicaprylyl Carbonate | Xanthan Gum |
| Squalane | Sodium Phytate |
| Butylene Glycol | Phenoxyethanol |
| B-silk protein | Ethylhexylglycerin |
| Cetearyl Olivate | |
| Sorbitan Olivate | |

Pick up was better for the silicone elastomer product and the product looks creamier and smoother. Product applied similarly, similar play time. Silk product dried down faster and had a powder dry down Silicone sample had an oilier dry down, did not seem to dry down. The silicone version was quite shiny on the skin. The b-silk seemed to fully absorb/adhere to the skin, dried down softer, and the slip stopped slipping.

### Primer #2

| | | | | | |
|---|---|---|---|---|---|
| | | | | 7.5% | |
| | % w/w | | | silicone | |
| Ingredient | range | Actuals: | 2% b- | elastomer | placebo |

| | | | | | |
|---|---|---|---|---|---|
| Water (Aqua) | 60-82 | | 67.765 | 69.762 | 69.753 |
| Carbomer | <1 | | 0.35 | 0.35 | 0.35 |
| Xanthan Gum | <1 | | 0.1 | 0.1 | 0.1 |
| Trisodium | <1 | | 0.3 | 0.3 | 0.3 |
| Ethylenediamine | | | | | |
| Disuccinate | | | | | |
| Glycerin | 3-5 | | 4 | 4 | 4 |
| Propanediol | 1-3 | | 2 | 2 | 2 |
| Caprylhydroxamic Acid | <3 | | 3 | 3 | 3 |
| 1,2-Hexanediol | <3 | | | | |
| Propanediol | <3 | | | | |
| Potassium Cetyl | .1-1 | | 0.5 | 0.5 | 0.5 |
| Phosphate | | | | | |
| Cetyl Alcohol | .5-2 | | 1.25 | 1.25 | 1.25 |
| Stearic Acid | <1 | | 0.9 | 0.9 | 0.9 |
| Glyceryl Stearate | <3 | | 2.25 | 2.25 | 2.25 |
| PEG-100 Sterarate | <3 | | | | |
| Coco- | <8 | | 7.75 | 7.75 | 7.75 |
| C9-12 Alkane | <8 | | | | |
| Caprylic/Capric | 4-9 | depending on | 7.75 | 4 | 7.75 |
| Triglyceride | | whether silk | | | |
| | | or silicone | | | |
| | | elastomer | | | |
| Aminomethyl Propanol | .05-1 | depending on | 0.085 | 0.088 | 0.097 |
| | | whether silk | | | |
| | | or silicone | | | |
| | | elastomer | | | |

silicone vs silicone-free with bsilk performed similarly.

### Shampoo

| | % w/w range | | |
|---|---|---|---|
| | | Silicone | |
| | | Elastomer | B-silk |
| Ingredient | Placebo | (3%) | (0.05%-1.0%) |
| Water (Aqua) | 41 | 37.7 | 39.7-40.6 |
| Acrylates Copolymer | 4 | 4 | 4 |
| Polyquat (Guar Hydroxypropyltrimonium Chloride) | 0.25 | 0.25 | 0.25 |
| Trisodium Ethylenediamine Disuccinate | 0.3 | 0.3 | 0.3 |
| Panthenol | 1 | 1 | 1 |
| Glycerin | 1 | 1 | 1 |
| Betaine | 1 | 1 | 1 |
| Sodium C14-16 Olefin Sulfonate | 49 | 49 | 49 |
| Sodium Cocoamphoacetate | | | |
| Cocamidopropyl Hydroxysultaine | | | |
| Propanediol | | | |
| Phenoxyethanol | | | |
| Ethylhexylglycerin | | | |
| Cetrimonium Chloride | 1 | 1 | 1 |
| Benzyl Alcohol | 1 | 1 | 1 |
| Methylchloroisothiazolinone | | | |
| Methylisothiazolinone | | | |
| Propylene Glycol | | | |
| Triethylene Glycol | | | |
| Citric Acid | 0.75 | 0.727 | 0.707 |
| b-silk | — | — | 0.05-1.0 |
| Divinyldimethicone/Dimethicone Copolymer | — | 3 | — |
| C12-13 Pareth-23 | | | |
| C12-13 Pareth-3 | | | |

Placebo Had a nice amount of suds. When combed through wet, there was normal friction. Had friction when drying, but that could be the lack of conditioner or any treatment product. Did not see any lifting, anti-frizz, smoothing properties.

Silicone Could definitely feel the slip when shampooing. Smaller bubbles during the wash compared to the placebo. Added friction to the hair when wet combing. Added friction when styled and dried.

B-silk Did not the super slip that silicone has when washing the hair. Friction was noticeable when wet combing. Once dried, hair seems less frizzy but felt very soft. The friction felt less and less during the day.

### Serum

| | % w/w range | | |
|---|---|---|---|
| | | Silicone | |
| | | Elastomer | B-silk |
| Ingredient | placebo | (0.75%) | (0.01-0.75%) |
| Water (Aqua) | 81.7 | 80.82 | 80.8-81.4 |
| Trisodium Ethylenediamine | 0.3 | 0.3 | 0.3 |
| Disuccinate | | | |
| Caprylhydroxamic Acid | 3 | 3 | 3 |
| 1,2-Hexanediol | | | |
| Propanediol | | | |
| Propanediol | 5 | 5 | 5 |
| Sodium Acrylates Copolymer | 1 | 1 | 1 |
| Lecithin | | | |
| Cetearyl Olivate | 2 | 2 | 2 |
| Sorbitan Olivate | | | |
| Brassica Glycerides | 1 | 1 | 1 |
| Caprylic/Capric Triglyceride | 3 | 3 | 3 |
| Coco-Caprylate/Caprate | 3 | 3 | 3 |
| C9-12 Alkane | | | |
| Citric Acid | 0.13 | 0.13 | 0.13 |
| b-silk | — | — | 0.1-0.75 |
| Dimethicone | — | 0.75 | — |
| Dimethicone/Vinyl Dimethicone | | | |
| Crosspolymer | | | |

Placebo vs Silicone Both applied similarly. The silicone version dried down smoother than the placebo and was not as tacky. Silicone version had a slightly more matte finish on the skin. Silicone version felt lighter on the skin (or maybe more absorbed?).

Placebo vs B-silk Both applied similarly. Dried down very similarly.

Silicone vs B-silk The b-silk added an extra cushiony feel on application. Otherwise, they were very similar. The b-silk version dried more matte than the others. B-silk was slightly more off-white, the other 2 were pure white. The b-silk version dried and absorbed quicker. Net-net, the b-silk added a richer feel during application and dried quicker to a nice, soft finish.

### SPF

| 1.5% b-silk | 2% linear silicones | 1.5% silicone elastomers | |
|---|---|---|---|
| 15/348-066 | 16/348-067 | 20/354-061 | % |
| Active Ingredient | Active Ingredient | Active Ingredient | |
| Zinc Oxide | Zinc Oxide | Zinc Oxide | 18.90% |
| Inactive Ingredients | Inactive Ingredients | Inactive Ingredients | |
| Aloe Barbadensis Leaf | Aloe Barbadensis Leaf | Aloe Barbadensis Leaf Juice | .01-5% |
| Aqua/Water | Aqua/Water | Water | 45-50% |
| b-Silk Protein | | | |
| Butyrospermum Parkii | Butyrospermum Parkii | Butyrospermum Parkii Nut | .01-5% |
| (Shea) Nut Extract | (Shea) Nut Extract | Extract | |
| Camellia Sinesis (Green | Camellia Sinesis (Green | Camellia Sinensis Leaf | .01-5% |
| Tea) Leaf Extract | Tea) Leaf Extract | | |
| Caprylic/Capric | Caprylic/Capric | Caprylic/Capric Triglyceride | 13-16% |
| Capryloyl Glycerin/Sebacic | Capryloyl Glycerin/Sebacic | Capryloyl | .1-10% |
| Acid Copolymer | Acid Copolymer | Glycerin/Sebacic Acid | |
| Caprylyl/Capryl Glucoside | Caprylyl/Capryl Glucoside | Caprylyl/Capryl Glucoside | .01-5% |
| Cetearyl Alcohol | Cetearyl Alcohol | Cetearyl Alcohol | 1-5% |
| Cetearyl Glucoside | Cetearyl Glucoside | Cetearyl Glucoside | .01-5% |
| Coco-caprate/caprylate | Coco-caprate/caprylate | Coco-caprate/caprylate | .01-5% |
| Coco-Glucoside | Coco-Glucoside | Coco-Glucoside | .1-10% |
| Coconut Alkanes | Coconut Alkanes | Coconut Alkanes | .01-5% |
| | Cyclohexasiloxane | | |
| | Cyclopentasiloxane | Cyclopentasiloxane | 1-5% |
| Diheptyl Succinate | Diheptyl Succinate | Diheptyl Succinate | .1-10% |
| | Dimethicone | | |
| | | Dimethicone/Vinyl | .01-5% |
| | | Dimethicone Crosspolymer | |
| Dipotassium Glycyrrhizate | Dipotassium Glycyrrhizate | Dipotassium Glycyrrhizate | .01-5% |
| Ethylhexylglycerin | Ethylhexylglycerin | Ethylhexylglycerin | .01-5% |
| Glycerin | Glycerin | Glycerin | 1-5% |
| Hydrolyzed Jojoba Esters | Hydrolyzed Jojoba Esters | Hydrolyzed Jojoba Esters | .01-5% |
| Isostearic Acid | Isostearic Acid | Isostearic Acid | .1-10% |
| Lecithin | Lecithin | Lecithin | .1-10% |
| Phenoxyethanol | Phenoxyethanol | Phenoxyethanol | .1-10% |
| Polyglyceryl-3 | Polyglyceryl-3 | Polyglyceryl-3 | .1-10% |
| Polyhydroxystearic Acid | Polyhydroxystearic Acid | Polyhydroxystearic Acid | .1-10% |
| Potassium Sorbate | Potassium Sorbate | Potassium Sorbate | .01-5% |
| Pyrus Malus (Apple) | Pyrus Malus (Apple) | Pyrus Malus (Apple) | .01-5% |
| Fruit Extract | Fruit Extract | Fruit Extract | |
| Sclerotium Gum | Sclerotium Gum | Sclerotium Gum | .01-5% |
| Sodium Benzoate | Sodium Benzoate | Sodium Benzoate | .01-5% |
| Sodium Phytate | Sodium Phytate | Sodium Phytate | .01-5% |
| Squalane | Squalane | Squalane | 1-5% |
| Tocopherol | Tocopherol | Tocopherol | .01-5% |
| Xanthan Gum | Xanthan Gum | Xanthan Gum | .01-5% |

### Leave-In Conditioner

Ingredients

| | |
|---|---|
| water | argania spinosa kernel oil |
| glycerin | hydroxyethylcellulose |
| cetearyl alcohol | cetyl glyceryl ether |
| caprylic/capric triglyceride | behentrimonium methosulfate |
| helianthus annuus (sunflower) | panthenol |
| seed oil | |
| propanediol | caprylhydroxamic acid |

### Review of Leave-In Conditioners

Viscosity: B-silk was much more flowable, keratin and empty formulas were similar in viscosity (thicker than b-silk).

Smell: No noticeable difference indicated

Application: During application keratin and empty formulas felt a bit more oily and slippery. B-silk formula had more resistance.

Dry down: The b-silk formula felt drier (less oily) and soft and slightly more volume. Keratin and empty formulas felt a bit more oily, free of tangles.

The b-silk formula did not leave hair as silky smooth and tangle free, makes the hair shaft feel textured.

Styling: Styling was not negatively affected

More fly-aways were seen in the untreated hair, very few fly aways were seen with the keratin and empty formulas. The number of fly aways for the b-silk protein was in between.

### Detangling Spray

Ingredients

| | |
|---|---|
| water | valine |
| glycerin | isoleucine |
| sodium PCA | proline |
| propanediol | threonine |
| argania spinosa kernel oil | histidine |
| arginine | phenylalanine |
| caprylhydroxamic acid | brassicamidopropyl |
| | dimethylamine |
| aspartic acid | pentylene glycol |
| PCA | polysorbate 20 |
| glycine | cetyl glyceryl ether |
| alanine | sodium lactate |
| serine | citric acid |

### Hydrogel Cream

Water
Glycerin
Coconut Alkanes
Pentylene Glycol

Hydroxyethyl Acrylate/Sodium
Acryloyldimethyl Taurate
Copolymer

sr-Spider Silk Protein
Acer Rubrum Bark Extract
Golden Birch Bark Extract
Black Spruce Bark Extract
Jack Pine Bark Extract
Hyaluronic Acid
Squalane
Coco-Caprylate/Caprate
Sodium Stearoyl Glutamate
Polyacrylate Crosspolymer-6

### Serum #3

Ingredients

| | |
|---|---|
| Water | proline |
| Pentylene Glycol | sodium hydroxide |
| spider silk protein | threonine |
| squalane | histidine |
| propanediol | phenylalanine |
| ceramide NP | biosaccharide gum-0 |
| ceramide AP | sucrose stearate |
| phytosphingosine | sodium lactate |
| sodium lauroyl lactylate | ammonium acryloyldimethyltaurate/vp |
| | copolymer |
| sodium pca | sodium stearoyl glutamate |
| sodium hyaluronate | citric acid |
| crosspolymer | |
| arginine | glycerin |
| aspartic acid | tetrasodium glutabmate diacetate |
| PCA | hydrogenated lecithin |
| glycine | polyglceryl-10 stearate |
| alanine | caprylhydroxamic acid |
| serine | cetyl glyceryl ether |
| valine | |
| isoleucine | |

### Rich Cream

Ingredients

| | |
|---|---|
| water | Phytosphingosine |
| propylheptyl caprylate | Cetyl Alcohol |
| Squalane | Caprylhydroxamic |
| | Acid |
| Pentylene Glycol | Potassium Cetyl |
| | Phosphate |
| Glycerin | polyacrylate |
| | crosspolymer-6 |
| Sunflower Seed Oil | carbomer |
| Cetearyl Olivate | |
| Propanediol | Maltodextrin |
| Sorbitan Olivate | tetrasodium |
| | glutamate diacetate |
| sr-Spider Silk Protein | cetyl glyceryl ether |
| Shea Butter | hydrogenated |
| | lecithin |
| Sodium Hyaluronate | polyglyceryl-10 |
| Crosspolymer | stearate |
| (Hyaluronic Acid) | |
| Ceramide NP | sucrose stearate |
| Ceramide AP | sodium hydroxide |
| Gentianaceae Leaf | |
| Extract | |

### Formulation Studies

B-Silk Protein powder as compared to Variati Protean S, was observed to be softer in feel (vs Variati feeling more like talc) and more translucent on application (vs Variati having a white cast). Additionally, B-Silk protein was a gel when used at 20% in water while the Variati turned to liquid. This give B-Silk an advantage for being versatile as a thickener for water phase.

### Foundation Study

Based on the Foundation Study, B-Silk Protein helped to enhance color development and use less of pigments. It helped for better coverage and spreadibility of the foundation. It helped with long wearing. It had the ability to absorb oil and make the product more matte when use at 10% level. The tests were done on hand, forearm, bioskin and actual wear for validation. The recombinant silk protein can be used in an amount of about 1%-10% for foundation.

The following formulations were analyzed:
1) Material: Wet & Wild Dewy Foundation (Control)
2) A=Wet & Wild+1% of B-Silk Protein
3) B=Wet & Wild+1% of Competition's Silk Protein
4) C=Cover FX Natural Finish Foundation
5) D=Saie Slip Tint
6) E=Wet & Wild+10% silk protein
7) F=(C )+5% silk protein
8) G=(D)+5% silk protein

The set of materials were checked on hand, foreharm, bioskin, and actual wear on face.

### 1) Control Vs. (A)

Better spreadibility
Sets Foundation quicker
Less Smudging
Color developed a bit darker
Better coverage
Less oily feel
Less radiant. Another attempt was made with 10% B-Silk protein and it had mattifying function at 10%.

### 2) Control Vs (B) (FIG. 1)

No significant difference in feel.
It made the product whiter.
   3) (A) vs. (C) CoverFX is a thick paste (FIG. 2)
(A) was like second skin smooth. (C) had high coverage almost like a concealer.

(A) vs (D) Saie was a sheer tint.

(F) vs C=Spread better. Color develops better (darker). Less glowing.

(G) vs D=Better coverage and less glowing.

(E) vs. Control=much darker and more matte.

Referring to FIG. 3, from control to A-G on BioSkin. With the addition of B-Silk Protein, the color developed further. Additionally, during washing, E was the hardest to clean off even with double cleansing due to 10% load of the B-Silk protein.

Referring to FIG. 4, the control foundation and (A) were also checked on face for actual wear. There was an immediate difference on coverage and feel. The side with 1% B-Silk Protein provided better coverage and has a powdery matte finish. It also felt less oily near the sides of nose area. When examined with beautyscope at 200×, it was also observed that with B-Silk protein, the coverage was better and less shiny. Additionally, after 8-hour wear, the cheeks with control and (A) were blotted with tissue paper. (A) showed better wearability over time.

### Lipstick Study

When the silk protein powder was added in the final formula, it acted as an oil absorber and can mattify the lipstick. It was observed that if the recombinant silk protein is processed in the product (melted), it can improve softening the structure and added sheen to the lipstick. When the B-Silk Protein was melted in the lipstick, it also enhanced the long-wear ability as it was harder to wash off. Whether added during manufacturing or top-added this powder, B-silk protein had the ability to deepen the color intensity and slightly give off blue tonality. Recombinant silk protein can be used in a lipstick composition in an amount of about 1-10%. At 10% or above, the formulation may be too soft. The following compositions were analyzed:
1. Formulation A=Material: Wet & Wild Lip Stick (Control)
2. Formulation B=Wet & Wild+10% of B-Silk Protein (melted in)
3. Formulation C=Mac
4. Formulation D=Mac+Silk Protein (Dip method)
5. Formulation E=Kosas
6. Formulation F=Kosas+Silk Protein (dip method)

Referring to FIG. 5, it was observed that with the B-Silk protein powder (Right), the color developed to be deeper and has a gloss to it. Without intending to be bound by theory, it is believed that this is due to the powder soften the structure of the lipstick a bit for easier application.

Formulations A & B were applied to bioskin then washed off with double cleansing and B stained better indicating long-wearing property with B-Silk Protein. FIG. 6 is a photo showing Formulation A vs. B (From Left to Right).

This was true for a higher end lipstick (MAC) as well. FIG. 7 shows a photo of formulation C vs D (L to R). The color of D is darker (bluer). Note: This was done via dipping lipstick to the powder on hand and mix without melting. The difference of pigment intensity still can be observed.

Referring to FIG. 8, another set of lipstick (Kosas) was also tested via dipping method E vs F (L to R). Interestingly it was observed that B-Silk Protein mattified the lipstick and increased the opacity.

### Mascara Study

B-Silk Protein powder was observed to improve volumizing properties with any type of mascaras. Without changing the formula, simply by addition of B-Silk Protein, it was observed that a lengthening mascara became a lengthening and volumizing mascara. B-Silk Protein powder can be applied prior to any mascaras to benefit from dramatic volumizing effects. B-Silk Protein Gel (20% solution) by itself can achieved a lengthening and defined eyelash look. Recombinant silk protein can be used in a mascara formulation in an amount of 1-10% in powder form or 20% minimum as a primer gel. The following compositions were tested:
1. Bare false eye lash
2. Control=(Wet & Wild Lengthening Mascara)
3. A=Control+10% powder via dipping method
4. B=Urban Decay Volumizing Mascara
5. B+=B+10% Powder via dipping method
6. C=Ilia Mascara
7. C+=C+10% powder via dipping method
8. Testing of B-Protein as a gel (20% in solution)

All of the mascaras benefited from addition of the B-Silk protein. Wet & Wild lengthening Mascara (control) with the addition of powder (A) had the most improvement. Referring to FIG. 9, the addition of a recombinant silk protein in accordance with the disclosure surprisingly turned a length mascara (control) into a volumizing mascara (A in FIG. 9).

Two other leading mascaras in the market were tested and the volumizing effect of B-silk was observed. B is a volumizing mascara by itself, and additional volume was observed with the addition of B-silk. C is a prestige lengthening mascara. The effects were less apparent compared to the mass market formula (A). This indicates that mass market mascaras with the addition of B-Silk protein can compete with prestige market just by adding the recombinant silk protein (FIG. 10).

Very little difference was observed amongst the various formulations when all included the B-silk (FIG. 11)

B-Silk Protein was also made into a 20% gel with water and applied to bare lashes as a potential primer. Surprisingly, it works just as well as a mass market lengthening mascara (FIG. 12).

However, when comparing primer with B-Silk protein+mascara vs B-Silk Protein powder straight addition, the powder still gave the most dramatic effect as the percent solids was higher than from the gel. (FIG. 13)

### Eyeshadow Study:

B-silk protein was observed to enhanced wearability by less smudging, and long-wearing. It gave a softer feel to eyeshadow and enhanced the brilliancy of formulations in which mica present in the formula. When B-Silk protein is used as a gel (20% in water), it intensified and enhanced the brilliancy of eyeshadow for finished products. When B-Silk protein gel was applied first as a primer, it resisted smudging even better. B-Silk protein gave different results when used as gel vs. powder by itself, making recombinant silk protein use in eyeshadow compositions quite versatile. An amount of about 1-10% recombinant silk protein can be used in eyeshadow compositions when used as a powder. A 20% minimum of the recombinant silk protein can be used for making a gel. The following formulations were tested:
1. A=Mac Pressed Eyeshadow Control
2. B=Mac Pressed Eyeshadow+Silk protein (dipping method)
3. C=A crushed into powder, add 10% of the B-Silk Protein powder and press.
4. D=MOB Beauty Control
5. E=MOB Beauty+Silk Protein (dipping method)

The pressed powder eyeshadows were evaluated on hand and bioskin with and without inclusion of the B-Silk protein. It was observed that B-Silk protein contributed to long-wearing, less smudging and less creasing when eyelids become oily (as a drop of oil has been placed on the powder eyeshadow application and blot off the see how much oil can be picked up by blotting.)

As illustrated in FIG. 14, less smudging on application was observed when B-silk protein was included in the formulation.

The same set of A & B were applied to bio skin and wash off with cleanser. There was much more staying power on B. (FIG. 15)

A drop of commercial hair oil was placed on the eye shadows and blended well. A tissue was used to blot to simulate potential eyelid creasing. It was observed that that the control was more likely to crease without the addition of B-Silk Protein. (FIG. 16)

The control sample was scrapped out and 10% of the silk protein powder was added and composition was repressed without additional binder. The shade turned to a beautiful teal with shimmer. (FIG. 17)

The process was also repeated with the 20% B-Silk protein gel to see any differences. The result is evident in the below photo that when used in the gel-format, it intensifies the chroma and looks brighter. (FIG. 18)

Another matte eyeshadow (D) was also tested. This particular eyeshadow did not work well with the B-Silk Protein powder. The formulation did not stay well. As a result, water was applied on hand first before application on both control and (E) which had been dipped with B-Silk Protein powder. A smudge test was performed and once again, (E) performed better. (FIG. 19)

### Equivalents and Scope

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended aspects.

In the aspects, articles such as “a,” “an,” and “the” may mean one or more than one unless indicated to the contrary or otherwise evident from the context. Aspects or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” and “consisting essential of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

What is claimed:

1. A cosmetic formulation comprising a recombinant silk protein formulated with a cosmetic base, wherein the cosmetic formulation has improved pigmentation.

2. The formulation of claim 1, wherein the recombinant silk protein is in a gel form.


SEQUENCE LISTING (37 sequences)

SEQ ID NO: 1 - protein, 5 aa, synthetic construct
SGAGG

SEQ ID NO: 2 - protein, 5 aa, synthetic construct
GSGAG

SEQ ID NO: 3 - protein, 5 aa, synthetic construct
GGSGA

SEQ ID NO: 4 - protein, 181 aa, Aliatypus gulosus
GAASSSSTIITTKSASASAAADASAAATASAASRSSANAAASAFAQSFSSILLESGYFCSIFGSSISSSYAAAIASAASRAAAESNGYTTHAYACAKAVASAVERVTSGADAYAYAQAISDALSHALLYTGRLNTANANSLASAFAYAFANAAAQASASSASAGAASASGAASASGAGSAS

SEQ ID NO: 5 - protein, 126 aa, Plectreurys tristis
GAGAGAGAGAGAGAGAGSGASTSVSTSSSSGSGAGAGAGSGAGSGAGAGSGAGAGAGAGGAGAGFGSGLGLGYGVGLSSAQAQAQAQAAAQAQAQAQAQAYAAAQAQAQAQAQAQAAAAAAAAAAA

SEQ ID NO: 6 - protein, 239 aa, Plectreurys tristis
GAAQKQPSGESSVATASAAATSVTSGGAPVGKPGVPAPIFYPQGPLQQGPAPGPSNVQPGTSQQGPIGGVGGSNAFSSSFASALSLNRGFTEVISSASATAVASAFQKGLAPYGTAFALSAASAAADAYNSIGSGANAFAYAQAFARVLYPLVQQYGLSSSAKASAFASAIASSFSSGTSGQGPSIGQQQPPVTISAASASAGASAAAVGGGQVGQGPYGGQQQSTAASASAAAATATS

SEQ ID NO: 7 - protein, 182 aa, Araneus gemmoides
GNVGYQLGLKVANSLGLGNAQALASSLSQAVSAVGVGASSNAYANAVSNAVGQVLAGQGILNAANAGSLASSFASALSSSAASVASQSASQSQAASQSQAAASAFRQAASQSASQSDSRAGSQSSTKTTSTSTSGSQADSRSASSSASQASASAFAQQSSASLSSSSSFSSAFSSATSISAV

SEQ ID NO: 8 - protein, 180 aa, Argiope aurantia
GSLASSFASALSASAASVASSAAAQAASQSQAAASAFSRAASQSASQSAARSGAQSISTTTTTSTAGSQAASQSASSAASQASASSFARASSASLAASSSFSSAFSSANSLSALGNVGYQLGFNVANNLGIGNAAGLGNALSQAVSSVGVGASSSTYANAVSNAVGQFLAGQGILNAANA

SEQ ID NO: 9 - protein, 199 aa, Deinopis spinosa
GASASAYASAISNAVGPYLYGLGLFNQANAASFASSFASAVSSAVASASASAASSAYAQSAAAQAQAASSAFSQAAAQSAAAASAGASAGAGASAGAGAVAGAGAVAGAGAVAGASAAAASQAAASSSASAVASAFAQSASYALASSSAFANAFASATSAGYLGSLAYQLGLTTAYNLGLSNAQAFASTLSQAVTGVGL

SEQ ID NO: 10 - protein, 171 aa, Trichonephila clavipes
GATAASYGNALSTAAAQFFATAGLLNAGNASALASSFARAFSASAESQSFAQSQAFQQASAFQQAASRSASQSAAEAGSTSSSTTTTTSAARSQAASQSASSSYSSAFAQAASSSLATSSALSRAFSSVSSASAASSLAYSIGLSAARSLGIADAAGLAGVLARAAGALGQ

SEQ ID NO: 11 - protein, 268 aa, Argiope trifasciata
GGAPGGGPGGAGPGGAGFGPGGGAGFGPGGGAGFGPGGAAGGPGGPGGPGGPGGAGGYGPGGAGGYGPGGVGPGGAGGYGPGGAGGYGPGGSGPGGAGPGGAGGEGPVTVDVDVTVGPEGVGGGPGGAGPGGAGFGPGGGAGFGPGGAPGAPGGPGGPGGPGGPGGPGGVGPGGAGGYGPGGAGGVGPAGTGGFGPGGAGGFGPGGAGGFGPGGAGGFGPAGAGGYGPGGVGPGGAGGFGPGGVGPGGSGPGGAGGEGPVTVDVDVSV

SEQ ID NO: 12 - protein, 420 aa, Trichonephila clavipes
GVSYGPGGAGGPYGPGGPYGPGGEGPGGAGGPYGPGGVGPGGSGPGGYGPGGAGPGGYGPGGSGPGGYGPGGSGPGGYGPGGSGPGGYGPGGSGPGGYGPGGYGPGGSGPGGSGPGGSGPGGYGPGGTGPGGSGPGGYGPGGSGPGGSGPGGYGPGGSGPGGFGPGGSGPGGYGPGGSGPGGAGPGGVGPGGFGPGGAGPGGAAPGGAGPGGAGPGGAGPGGAGPGGAGPGGAGPGGAGGAGGAGGSGGAGGSGGTTIIEDLDITIDGADGPITISEELPISGAGGSGPGGAGPGGVGPGGSGPGGVGPGGSGPGGVGPGGSGPGGVGPGGAGGPYGPGGSGPGGAGGAGGPGGAYGPGGSYGPGGSGGPGGAGGPYGPGGEGPGGAGGPYGPGGAGGPYGPGGAGGPYGPGGEGGPYGP

SEQ ID NO: 13 - protein, 376 aa, Latrodectus hesperus
GINVDSDIGSVTSLILSGSTLQMTIPAGGDDLSGGYPGGFPAGAQPSGGAPVDFGGPSAGGDVAAKLARSLASTLASSGVFRAAFNSRVSTPVAVQLTDALVQKIASNLGLDYATASKLRKASQAVSKVRMGSDTNAYALAISSALAEVLSSSGKVADANINQIAPQLASGIVLGVSTTAPQFGVDLSSINVNLDISNVARNMQASIQGGPAPITAEGPDFGAGYPGGAPTDLSGLDMGAPSDGSRGGDATAKLLQALVPALLKSDVFRAIYKRGTRKQVVQYVTNSALQQAASSLGLDASTISQLQTKATQALSSVSADSDSTAYAKAFGLAIAQVLGTSGQVNDANVNQIGAKLATGILRGSSAVAPRLGIDLS

SEQ ID NO: 14 - protein, 200 aa, Argiope trifasciata
GAGYTGPSGPSTGPSGYPGPLGGGAPFGQSGFGGSAGPQGGFGATGGASAGLISRVANALANTSTLRTVLRTGVSQQIASSVVQRAAQSLASTLGVDGNNLARFAVQAVSRLPAGSDTSAYAQAFSSALFNAGVLNASNIDTLGSRVLSALLNGVSSAAQGLGINVDSGSVQSDISSSSSFLSTSSSSASYSQASASSTS

SEQ ID NO: 15 - protein, 357 aa, Uloborus diversus
GASAADIATAIAASVATSLQSNGVLTASNVSQLSNQLASYVSSGLSSTASSLGIQLGASLGAGFGASAGLSASTDISSSVEATSASTLSSSASSTSVVSSINAQLVPALAQTAVLNAAFSNINTQNAIRIAELLTQQVGRQYGLSGSDVATASSQIRSALYSVQQGSASSAYVSAIVGPLITALSSRGVVNASNSSQIASSLATAILQFTANVAPQFGISIPTSAVQSDLSTISQSLTAISSQTSSSVDSSTSAFGGISGPSGPSPYGPQPSGPTFGPGPSLSGLTGFTATFASSFKSTLASSTQFQLIAQSNLDVQTRSSLISKVLINALSSLGISASVASSIAASSSQSLLSVSA

SEQ ID NO: 16 - protein, 32 aa, Euprosthenops australis
GGQGGQGQGRYGQGAGSSAAAAAAAAAAAAAA

SEQ ID NO: 17 - protein, 42 aa, Tetragnatha kauaiensis
GGLGGGQGAGQGGQQGAGQGGYGSGLGGAGQGASAAAAAAAA

SEQ ID NO: 18 - protein, 42 aa, Argiope aurantia
GGYGPGAGQQGPGSQGPGSGGQQGPGGLGPYGPSAAAAAAAA

SEQ ID NO: 19 - protein, 46 aa, Deinopis spinosa
GPGGYGGPGQQGPGQGQYGPGTGQQGQGPSGQQGPAGAAAAAAAAA

SEQ ID NO: 20 - protein, 42 aa, Trichonephila clavipes
GPGGYGLGQQGPGQQGPGQQGPAGYGPSGLSGPGGAAAAAAA

SEQ ID NO: 21 - protein, 174 aa, Deinopis spinosa
GAGYGAGAGAGGGAGAGTGYGGGAGYGTGSGAGYGAGVGYGAGAGAGGGAGAGAGGGTGAGAGGGAGAGYGAGTGYGAGAGAGGGAGAGAGAGAGAGAGAGSGAGAGYGAGAGYGAGAGAGGVAGAGAAGGAGAAGGAGAAGGAGAAGGAGAGAGAGSGAGAGAGGGARAGAGG

SEQ ID NO: 22 - protein, 149 aa, Latrodectus hesperus
GGGYGRGQGAGAGVGAGAGAAAGAAAIARAGGYGQGAGGYGQGQGAGAAAGAAAGAGAGGYGQGAGGYGRGQGAGAGAGAGAGARGYGQGAGAGAAAGAAASAGAGGYGQGAGGYGQGQGAGAAAGAAASAGAGGYGQGAGGYGQGQGA

SEQ ID NO: 23 - protein, 161 aa, Trichonephila clavipes
GAGAGGAGYGRGAGAGAGAAAGAGAGAAAGAGAGAGGYGGQGGYGAGAGAGAAAAAGAGAGGAAGYSRGGRAGAAGAGAGAAAGAGAGAGGYGGQGGYGAGAGAGAAAAAGAGSGGAGGYGRGAGAGAAAGAGAAAGAGAGAGGYGGQGGYGAGAGAAAAA

SEQ ID NO: 24 - protein, 186 aa, Nephilengys sp.
GAGAGVGGAGGYGSGAGAGAGAGAGAASGAAAGAAAGAGAGGAGGYGTGQGYGAGAGAGAGAGAGGAGGYGRGAGAGAGAGAGGAGGYGAGQGYGAGAGAGAAAAAGDGAGAGGAGGYGRGAGAGAGAGAAAGAGAGGAGGYGAGQGYGAGAGAGAAAGAGAGGAGGYGAGQGYGAGAGAGAAAAA

SEQ ID NO: 25 - protein, 198 aa, Uloborus diversus
GAGAGYRGQAGYIQGAGASAGAAAAGAGVGYGGQAGYGQGAGASAGAAAAAGAGAGRQAGYGQGAGASAGAAAAGAGAGRQAGYGQGAGASAGAAAAGADAGYGGQAGYGQGAGASAGAAASGAGAGYGGQAGYGQGAGASAGAAAAGAGAGYLGQAGYGQGAGASAGAAAGAGAGYGGQAGYGQGTGAAASAAASSA

SEQ ID NO: 26 - protein, 198 aa, Uloborus diversus; identical to SEQ ID NO: 25

SEQ ID NO: 27 - protein, 190 aa, Araneus ventricosus
GGQGGQGGYGGLGSQGAGQGGYGAGQGAAAAAAAAGGAGGAGRGGLGAGGAGQGYGAGLGGQGGAGQAAAAAAAGGAGGARQGGLGAGGAGQGYGAGLGGQGGAGQGGAAAAAAAAGGQGGQGGYGGLGSQGAGQGGYGAGQGGAAAAAAAAGGQGGQGGYGGLGSQGAGQGGYGGRQGGAGAAAAAAAA

SEQ ID NO: 28 - protein, 166 aa, Dolomedes tenebrosus
GGAGAGQGSYGGQGGYGQGGAGAATATAAAAGGAGSGQGGYGGQGGLGGYGQGAGAGAAAAAAAAAGGAGAGQGGYGGQGGQGGYGQGAGAGAAAAAAGGAGAGQGGYGGQGGYGQGGGAGAAAAAAAASGGSGSGQGGYGGQGGLGGYGQGAGAGAGAAASAAAA

SEQ ID NO: 29 - protein, 177 aa, Nephilengys sp.
GGAGQGGYGGLGGQGAGAAAAAAGGAGQGGYGGQGAGQGAAAAAASGAGQGGYEGPGAGQGAGAAAAAAGGAGQGGYGGLGGQGAGQGAGAAAAAAGGAGQGGYGGLGGQGAGQGAGAAAAAAGGAGQGGYGGQGAGQGAAAAAAGGAGQGGYGGLGSGQGGYGRQGAGAAAAAAAA

SEQ ID NO: 30 - protein, 174 aa, Nephilengys sp.
GGAGQGGYGGLGGQGAGAAAAAAGGAGQGGYGGQGAGQGAAAAAASGAGQGGYGGPGAGQGAGAAAAAAGGAGQGGYGGLGGQGAGQGAGAAAAAAGGAGQGGYGGQGAGQGAAAAAAGGAGQGGYGGLGSGQGGYGGQGAGAAAAAGGAGQGGYGGLGGQGAGQGAGAAAAAA

SEQ ID NO: 31 - protein, 5 aa, synthetic construct
SGGQQ

SEQ ID NO: 32 - protein, 5 aa, synthetic construct
GAGQQ

SEQ ID NO: 33 - protein, 6 aa, synthetic construct
GQGOPY

SEQ ID NO: 34 - protein, 4 aa, synthetic construct
AGQQ

SEQ ID NO: 35 - protein, 11 aa, synthetic construct
  MOD_RES 7: note = X is SEQ ID NO: 31, SEQ ID NO: 32, SEQ ID NO: 33, or SEQ ID NO: 34
GGYGPGXGPSA

SEQ ID NO: 36 - protein, 944 aa, synthetic construct
GGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAAGGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGPSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAA

SEQ ID NO: 37 - protein, 314 aa, synthetic construct
GGYGPGAGQQGPGSGGQQGPGGQGPYGSGQQGPGGAGQQGPGGQGPYGPGAAAAAAAAAGGYGPGAGQQGPGGAGQQGPGSQGPGGQGPYGPGAGQQGPGSQGPGSGGQQGPGGQGPYGSAAAAAAAAAGGYGPGAGQRSQGPGGQGPYGPGAGQQGPGSQGPGSGGQQGPGGQGPYGPSAAAAAAAAGGYGPGAGQQGPGSQGPGSGGQQGPGGQGPYGPGAAAAAAAVGGYGPGAGQQGPGSQGPGSGGQQGPGGQGPYGPSAAAAAAAAGGYGPGAGQQGPGSQGPGSGGQQGPGGQGPYGPSAAAAAAAA